(19) United States
(12) Patent Application Publication
(10) Pub. No.: US 2023/0143748 A1
(43) Pub. Date: May 11, 2023

(54) SPATIALLY-AWARE INTERACTIVE SEGMENTATION OF MULTI-ENERGY CT DATA

(71) Applicant: MARS BIOIMAGING LIMITED, Christchurch (NZ)

(72) Inventors: Philip Howard BUTLER, Christchurch (NZ); Anthony Philip Howard BUTLER, Christchurch (NZ); Niels J A DE RUITER, Christchurch (NZ); Robert LINDEMAN, Christchurch (NZ); Praveenkumar KANITHI, Abu Dhabi (AE)

(21) Appl. No.: 17/996,860

(22) PCT Filed: Apr. 22, 2021
(86) PCT No.: PCT/IB2021/053350

§ 371 Date: Oct. 21, 2022

(30) Foreign Application Priority Data
Apr. 24, 2020 (NZ) 763883

(51) Int. Cl. G06T 7/174; G06N 20/20; G06V 10/44; G06T 7/194; G06V 10/26; G06T 7/00

(52) U.S. Cl. CPC G06T 7/174; G06N 20/20; G06V 10/457; G06T 7/194; G06V 10/267; G06T 7/0012; G06T 2207/20081; G06T 2207/20076; G06V 2201/031; G06T 2207/30096; G06T 2207/30012; G06T 2207/10084

(57) ABSTRACT

Segmentation of multi-energy CT data, including data in three or more energy bands. A user is enabled to input one or more region indicators in displayed CT data. Probability maps are generated and may be refined using distance metrics, which may include geodesic and Euclidean distance metrics. Segmentation may be based on the probability maps and/or refined probability maps. Segmentation of medical image data is also disclosed.

### CROSS-REFERENCE TO RELATED DISCLOSURES

The present disclosure is a National Phase Entry of International Application No. PCT/IB2021/053350 filed on Apr. 22, 2021 and claims the benefit of NZ Application No. 763883 filed Apr. 24, 2020, the disclosures of which being incorporated herein in entirety.

### FIELD OF INVENTION

This invention relates to the segmentation of data produced using a multi-energy computed tomography (CT) scanning system.

### BACKGROUND TO THE INVENTION

Multi-energy computed tomography (CT) is an x-ray imaging modality which produces 3D images of the inside of objects. CT scanners use polychromatic x-ray sources which emit a full rainbow, or spectrum, of x-rays with various ‘colours’ (x-ray energies). In regular CT there is no distinction made between the different energies of x-rays. However, x-rays are absorbed differently by different materials in the body, and absorption also depends on x-ray energy. Multi-energy CT measures the absorption of x-rays in different energy ranges. Using the differences in x-ray absorption in these energy ranges it is possible to discriminate between (identify) and quantify various materials in a scanned subject.

Interactive image segmentation is a process of partitioning an image into disjoint and meaningful regions with the help of user guidance. Image segmentation in general plays an important role in a wide range of medical imaging applications and analysis tasks.

For example, image segmentation may allow the separation of trabecular and cortical bone to evaluate bone health, quantifying nano particles in a particular organ, tumour segmentation, region specific material quantification, etc.

However, accurate and automatic segmentation of image data produced by a multi-energy CT system remains a challenging problem. Some solutions for dual-energy CT image data are known.

However, little to no labelled data, that is required for designing automatic segmentation solutions, is available for the segmentation of data produced using multi-energy CT. The use of existing segmentation methods fails to provide satisfactory results as they are unable to efficiently process the large amount of data produced by a multi-energy CT system.

Several pre-clinical studies have shown that multi-energy CT is able to differentiate various types of tissues in the human body, as well as different contrast agents commonly used in CT imaging. However, present interactive segmentation methods are not designed to take advantage of the additional information provided by multi-energy CT images.

Outside of the field of multi-energy CT, some known segmentation methods use superpixels to perform fewer computations. However, most superpixel driven methods assume the input image to be a single channel or an RGB image and use only low-level features to segment the image into the foreground and background. If superpixel generation is not accurate, it may lead to inaccurate boundaries.

Region-based methods use preliminary features such as histograms and mean intensities to represent superpixels. Due to the multi-channel nature of multi-energy CT datasets (e.g. the Applicant's datasets currently range from 4 to 8 channels), following the same strategy leads to an increase in the histogram length by the number of bins per channel times the additional number of channels, which in turn may cause curse of dimensionality problems.

For the purposes of this specification, the term “multi-energy CT” refers to CT with 3 or more energy ranges or bins. This is distinct from dual-energy CT which uses only two energies.

It would be an advantage to develop an image segmentation method that produces more accurate segmentation of multi-energy CT image data sets or at least addresses some of the above issues.

### OBJECT OF THE INVENTION

It is an object of the invention to provide a computer implemented segmentation method suitable for use with data produced using a multi-energy CT system.

Alternatively, it is an object of the invention to provide a computer implemented segmentation method using probability maps and/or refined probability maps.

Alternatively, it is an object of the invention to at least provide the public with a useful choice.

### SUMMARY OF THE INVENTION

A computer-implemented method for segmentation of multi-energy CT data including data in three or more energy bands may include: receiving in memory the multi-energy CT data; displaying the multi-energy CT data to a user; receiving user input of one or more region indicators for the displayed data; based on the region indicators, generating one or more pixel probability maps; and based on the one or more pixel probability maps, segmenting the multi-energy CT data.

At least one of the one or more pixel probability maps may be generated using a decision tree learning model. The decision tree learning model may be a bootstrap aggregated decision tree learning model. The decision tree learning model may be a random forest learning model.

At least one of the one or more probability maps may be refined before segmenting the multi-energy CT data. Refining may include adjusting the probability values for at least some pixels based on one or more distance metrics. For a particular pixel, the one or more distance metrics may be indicative of one or more distances between that particular pixel and one or more of the region indicators. The one or more distances may include a Euclidean distance. The one or more distances may include a Geodesic distance.

The refinement of the one or more probability maps may be repeated.

Each pixel probability map may define, for each pixel, a probability of that pixel being in a data region.

Segmenting the CT data may include labelling pixels as belonging to different regions. Segmenting the CT data may include segmenting the CT data into two regions.

Segmenting the CT data may be performed using a conditional random field segmentation method.

Segmenting the CT data may include: performing an initial segmentation of the CT data, including identifying one or more boundaries between regions; refining the initial segmentation of the CT data by further analysis of data around the identified one or more boundaries. A band of data may be defined around each identified boundary, wherein the further analysis is of data within the defined band.

The initial segmentation may be performed using a conditional random field segmentation method with a first neighbour connectivity, and the refinement of the initial segmentation may be performed using a conditional random field segmentation method with a second neighbour connectivity higher than the first neighbour connectivity.

The initial segmentation may be performed using an alpha-expansion algorithm, and the refinement of the initial segmentation is performed by creating a dense graph using pixels around the identified one or more boundaries and refining the initial segmentation using alpha-expansion optimization.

A computer-implemented method for segmentation of medical image data may include: receiving in memory the medical image data; displaying the medical image data to a user; receiving user input of one or more region indicators for the displayed data; based on the region indicators, generating one or more pixel probability maps; refining at least one of the one or more probability maps by adjusting probability values for at least some pixels based on two or more distance metrics, wherein, for a particular pixel, the two or more distance metrics are indicative of two or more distances between that particular pixel and one or more of the region indicators, and wherein the two or more distances include a Euclidean distance and a geodesic distance; and based on the refined pixel probability maps, segmenting the medical image data.

At least one of the one or more pixel probability maps may be generated using a decision tree learning model. The decision tree learning model may be a bootstrap aggregated decision tree learning model. The decision tree learning model may be a random forest learning model.

The refinement of the one or more probability maps may be repeated.

Each pixel probability map may define, for each pixel, a probability of that pixel being in a data region.

Segmenting the image data may include labelling the pixels as belonging to different regions. Segmenting the image data may include segmenting the image data into two regions.

Segmenting the image data may be performed using a conditional random field segmentation method.

Segmenting the image data may include: performing an initial segmentation of the image data, including identifying one or more boundaries between regions; refining the initial segmentation of the image data by further analysis of data around the identified one or more boundaries. A band of data may be defined around each identified boundary, wherein the further analysis is of data within the defined band.

The initial segmentation may be performed using a conditional random field segmentation method with a first neighbour connectivity, and the refinement of the initial segmentation is performed using a conditional random field segmentation method with a second neighbour connectivity higher than the first neighbour connectivity.

The initial segmentation may be performed using an alpha-expansion algorithm, and the refinement of the initial segmentation is performed by creating a dense graph using pixels around the identified one or more boundaries and refining the initial segmentation using alpha-expansion optimization.

The medical image data may include data captured from a human or animal subject.

The medical image data may be three dimensional medical image data.

Any of the above methods may be used for 3D segmentation, for example by: generating a plurality of 2D slices from the 3D data; selecting one of the 2D slices as a start slice, wherein the received region indicators are in the start slice and the segmentation produces a segmented start slice; propagating data to one or more adjacent further slices; based in part on the propagated data, segmenting the one or more further slices; repeating the propagating of data and segmentation of further slices until the segmented start slice and segmented further slices encompass a desired 3D space; and combining the segmented slices to form segmented 3D data.

The propagated data may include propagated data based on the region indicators.

The propagated data may include morphological data.

The segmented slices or the segmented 3D data may be processed to smooth transitions between slices.

A multi-energy CT data segmentation system may include: memory arranged to store multi-energy CT data including data in three or more energy bands; a display arranged to display the multi-energy CT data to a user; a user input device arranged for user input of one or more region indicators for the displayed data; and a processor arranged to: based on the region indicators, generate one or more pixel probability maps; based on the one or more pixel probability maps, segment the multi-energy CT data.

A multi-energy CT system may include: a multi-energy CT scanner configured to scan a subject to produce multi-energy CT data including data in three or more energy bands; memory arranged to store the multi-energy CT data; a display arranged to display the multi-energy CT data to a user; a user input device arranged for user input of one or more region indicators for the displayed data; and a processor arranged to: based on the region indicators, generate one or more pixel probability maps; based on the one or more pixel probability maps, segment the multi-energy CT data.

A medical image segmentation system, may include: memory arranged to store medical image data; a display arranged to display the medical image data to a user; a user input device arranged for user input of one or more region indicators for the displayed data; and a processor arranged to: based on the region indicators, generate one or more pixel probability maps; refine at least one of the one or more probability maps by adjusting probability values for at least some pixels based on two or more distance metrics, wherein, for a particular pixel, the two or more distance metrics are indicative of two or more distances between that particular pixel and one or more of the region indicators, and wherein the two or more distances include a Euclidean distance and a geodesic distance; and based on the refined pixel probability maps, segment the multi-energy CT data.

In further embodiments a computer-implemented method for segmentation of an image produced using a multi-energy CT system using three or more energy bands, may include:
a) scanning an object using a multi-energy CT system using three or more energy bands;
b) storing the image data set of a) on a storage medium;
c) sending image data to be displayed on a graphical user interface;
d) receiving a plurality of strokes/markings generated by user input to the digital image to label image sections as a foreground or background;
e) generating a pixel probability map from the input of d) using random forests;
f) refining the probability map of e) using geodesic distance cues;
g) refining the probability map of e) using Euclidean distance cues;
h) combining the refined probability maps off) and g) to generate a final probability map;
i) generating final segmented image data from h) using conditional random fields; and
j) presenting the segmented image to an interface.

The refinement of the probability map using geodesic distance cues may include solving a set of non-linear Eikonal equations using Fast Marching Methods.

The refinement of the probability map using Euclidean distance cues may include using pixel location information to predict whether a pixel is a foreground or background pixel.

The use of conditional random fields may include a first step of labelling individual pixels and a second step of smoothing boundaries between pixels with different labels.

The step of generating final segmented data may include labelling the pixels as foreground or background.

Labelling the pixels as foreground or background may include:
a) using an alpha-expansion algorithm to segment all the image data; and
b) creating a dense graph using pixels around segmented image boundaries and refining the result using alpha-expansion optimization.

The method may include repeating steps d)-i) to obtain improved segmentation accuracy.

A system for segmentation of an image produced using a multi-energy CT system using three or more energy bands, may include:
a) a means for scanning an object using a multi-energy CT system to produce a set of image data;
b) a means for storing the set of image data;
c) a means for sending the set of image data to a graphical user interface;
d) a means for visually displaying the image data;
e) a means for receiving a plurality of strokes/markings generated by user input;
f) a means for generating a pixel probability map from the input of e) using random forests;
g) a means for refining the probability map of f) using geodesic distance cues;
h) a means for refining the probability map of f) using Euclidean distance cues;
i) a means for combining the refined probability maps of g) and h) to generate a final probability map;
j) a means for generating final segmented image data from h) using conditional random fields; and
k) a means for presenting the segmented image to an interface.

A system for segmentation of an image produced using a multi-energy CT system using three or more energy bands, may include:
a) a multi-energy CT scanning system for scanning an object to produce a set of image data;
b) a data storage medium for storing the set of image data;
c) a transmitter for sending the set of image data to a graphical user interface;
d) a graphical user interface for visually displaying the image data;
e) a processor for receiving a plurality of strokes/markings generated by user input;
f) a processor for generating a pixel probability map from the input of e) using random forests;
g) a processor for refining the probability map off) using geodesic distance cues;
h) a processor for refining the probability map off) using Euclidean distance cues;
i) a processor for combining the refined probability maps of g) and h) to generate a final probability map;
j) a processor for generating final segmented image data from h) using conditional random fields; and
k) an interface for presenting the segmented image to a user.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 shows the method of spatial aware interactive image segmentation of the input image using two stage conditional random fields for a single 2D slice in one embodiment of the invention;

FIG. 2 shows the computer implemented method of interactive image segmentation of FIG. 1 in one embodiment of the invention;

FIG. 3 shows an illustration of the construction of the augmented image for an RGB image in one aspect of the invention;

FIG. 4A shows probability map generation of the input image using user scribbles where (a) shows a sample image with user input, where dark and light scribbles represent foreground and background respectively, (b) foreground probability map using random forests and (c) background probability map using random forests;

FIG. 4B shows a visual comparison of the foreground probability maps generated by random forest and online random forest during multiple iterations (a) and (d), a sample image with use input during first and second iterations (b) and (e), foreground probability map using random forest (c) and (f) foreground probability map using online random forest;

FIG. 5 shows an example of refining probability maps using geodesic distance cues with (a) showing a sample image with user input, (b) a foreground probability map generated using random forests and (c) refined foreground probability map using geodesic distance transform;

FIG. 6 shows an example of refining initial probability maps using Euclidean distance cues with (a) showing a sample image with user input, (b) a foreground probability map generated using random forests, (c) Euclidean distance map calculated using scribble location and (d) refined foreground probability map using the Euclidean distance map in (c);

FIG. 7 shows an illustration of refining probability maps using Euclidean and geodesic distance cues with (a) showing a sample image with use input, (b) foreground probability maps generated using random forests, (c) geodesic distance transform for the probability map in (b), (d) refined probability map using a Euclidean distance map and (e) a final probability map after combining Euclidean and geodesic distance cues;

FIG. 8 shows a segmentation output from the first stage of CRF (a) and the cut dividing foreground and background pixels (b);

FIG. 9 shows a visual comparison of the segmentation results by the proposed approach compared with a bag of features method using lamb and knee data sets;

FIG. 10 shows segmentation results for a sheep femur dataset obtained from probability maps at different stages of refinement;

FIG. 11 shows segmentation results for a human CT dataset obtained from probability maps at different stages of refinement;

FIG. 12 shows segmentation results for a tibia plateau dataset obtained from probability maps at different stages of refinement;

FIG. 13 shows a visual comparison of the segmentation output during stage one and stage two of the proposed algorithm of FIGS. 1 and 2;

FIG. 14 shows a representation of one embodiment of a system for implementing any one or more of the methods shown in FIG. 1 or FIG. 2;

FIG. 15 shows a representation of an alternative embodiment of a system for implementing any one or more of the methods shown in FIG. 1 or FIG. 2;

FIG. 16 shows a workflow for volume segmentation using a slice-based approach

FIG. 17 shows a comparison of slice-wise and whole-volume three dimensional segmentations; and

FIG. 18 shows a quantitative comparison of the segmentations of FIG. 17.

### BRIEF DESCRIPTION OF PREFERRED EMBODIMENTS OF THE INVENTION

The invention disclosed herein relates mainly to the interactive segmentation of multi-energy CT data sets. The invention also relates to one or more CT scanning systems, software facilities, computer program products, computer systems or computer implemented methods for the interactive segmentation of multi-energy CT data sets. Certain aspects relate to interactive segmentation of medical image data.

The Applicant's segmentation method may be used, for example, for segmenting or isolating a particular region in multi-energy CT data. In CT scans on human or animal subjects, the region to be isolated may be a particular organ, part of an organ, bone, part of a bone, tumour or other anatomical region. Other types of subject may also be scanned, including e.g. inanimate samples, rock, wood, luggage, bags or any other scanning subject.

The method described below discloses one example of an interactive image segmentation method designed for use with images and image datasets produced using a multi-energy CT scanning system that uses three or more energy bands.

In addition to proper utilization of the spectral information produced by the multi-energy CT scanner, the proposed method may take advantage of spatial information provided by user input, and may provide a smooth segmentation boundary. The method may also be implemented as a software tool and integrated into multi-energy CT data visualisation applications that are designed to support multi-channel datasets.

Following user input (e.g. in the form of scribbles or markings), the example method described below may be divided into three major steps: i) generating probability maps (e.g. using a random forest method), ii) modifying or refining these probability maps (e.g. using Euclidean and geodesic distance cues to embed spatial awareness), and iii) image segmentation (e.g. with the help of a two stage CRF model for smooth and accurate object boundaries).

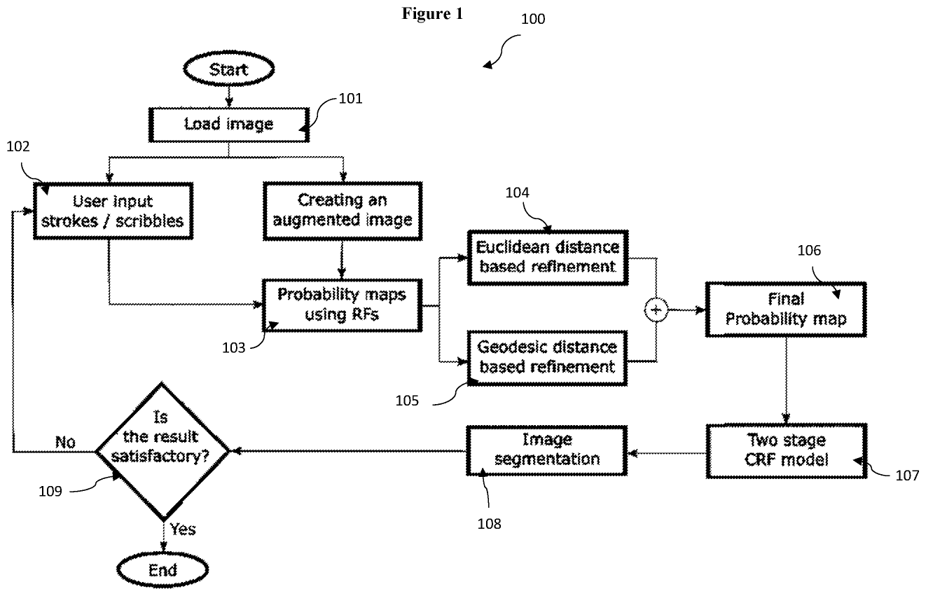

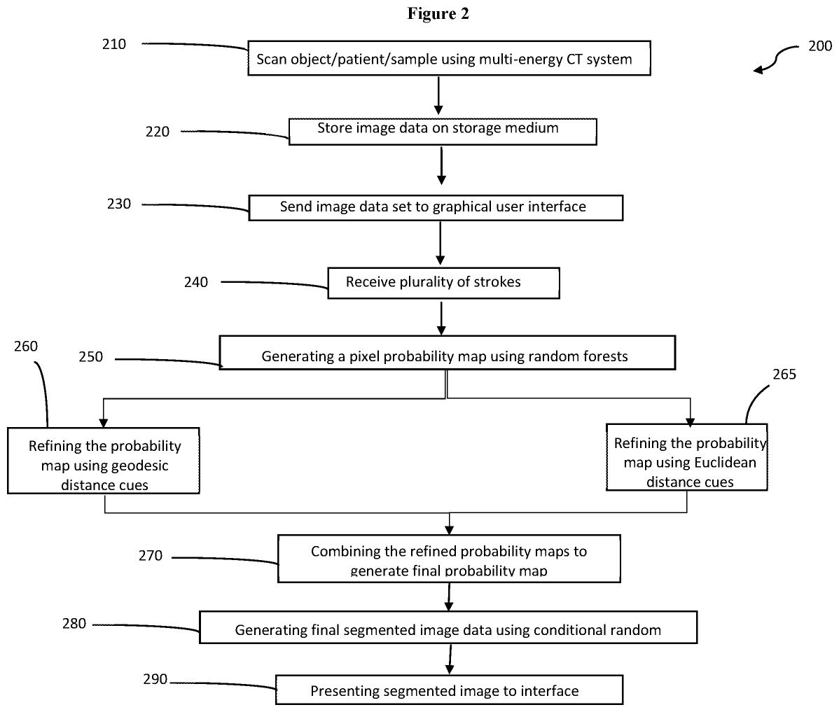

One method 100 of the proposed spatially-aware interactive image segmentation method for a single 2D slice is illustrated in FIG. 1. FIG. 2 outlines the method as a series of computer implemented steps.

These methods 100 and 200 are designed to be closed loops and can be iteratively refined or repeated (including repetition of user input, refinement of probability maps and/or segmentation) to improve the segmentation.

The method starts by loading a data set produced using a multi-energy CT system 101 and labelling pixels within the foreground and the background objects using mouse strokes/scribbles 102. For the implementation of interactive segmentation, the image is displayed to a user. The user inputs one or more region indicators, e.g. a set of strokes 102 via any suitable user input, including e.g. touch screens, pointer devices, computer mouse etc., to indicate the pixels that belong to a particular region or class. In many embodiments, there may be two regions which may be considered ‘foreground’ and ‘background’. Strokes, scribbles or markings may be entered onto an image on a graphical interface by an informed user to indicate foreground and background regions in an image. Information from these labelled pixels is used to generate probability maps/likelihood maps. This may be done using a Random Forest (RF) method 103.

At this stage, this information only consists of the visual properties of the data. To refine these probability maps, in other words, to make them spatially aware, the location of the labelled pixels corresponding to both the foreground and the background may be used. Refinement may use a Euclidean distance-based refinement 104 and/or a geodesic distance-based refinement 105, and a combination of at least Euclidean and geodesic distance metrics for the refinement may be advantageous as described below. Other types of refinement of the probability maps may be used. Further, refinement may be repeated in order to improve the quality of the resulting probability map. Probability maps or refinements to probability maps may be based on any suitable data, including the original CT data, any subset of the original data (e.g. isolated channels in the original CT data), any data derived from the original data (e.g. processed CT data, probability maps etc.), user input data etc. Refinement of the probability map results in a refined probability map. As such, the refinement may be repeated, or further refinements performed on the refined probability map, as required to produce a probability map that will support acceptable segmentation.

Step 105 refines the originally generated maps to be spatially localized so that there will not be any falsely segmented image patches away from the original object to create a final probability map 106.

A two-stage conditional random fields (CRF) model 107 may be used to perform the segmentation. The first stage involves segmenting the entire image using the refined probability maps, and four neighbourhood pixel connections (left, right, up, and down). However, having just four connections often results in jagged object boundaries. To smooth the boundaries, a second stage CRF may be employed around the initially segmented object boundaries with much denser connections. As shown in FIG. 1, if the user is not happy with the final segmentation result 108, more scribbles can be added to improve the result 109. The further user scribbles may be used to further refine the probability maps and resulting segmentation.

Probability Map Generation

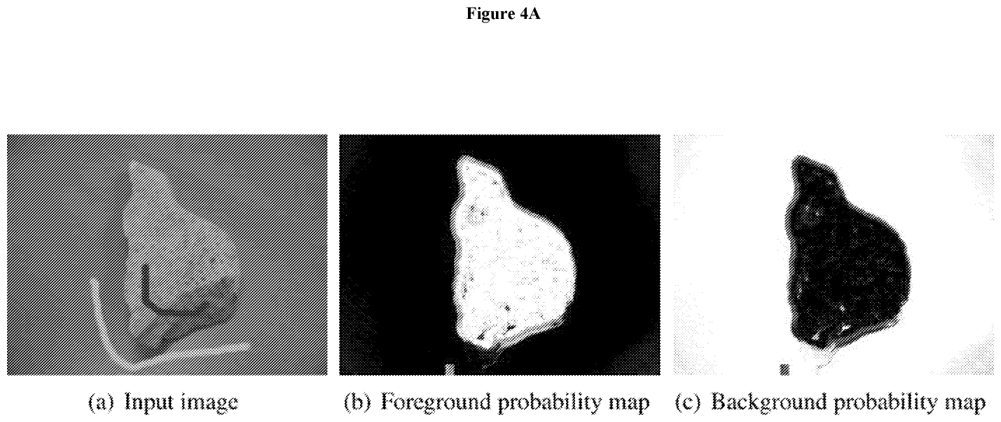

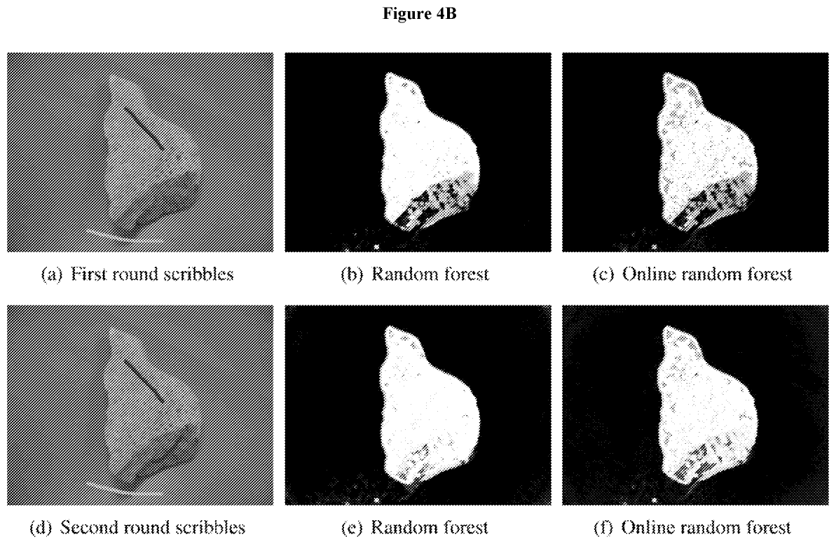

Probability maps represent how likely each pixel is to belong to a region (e.g. either foreground or background) as shown in FIG. 4A. These are also called likelihood maps. Based on the information gathered from the user-input seed points, all the remaining pixels are assigned a probability of how likely they are to belong to the foreground and background.

In the literature, the standard approach to generate these maps is to use Gaussian models using grayscale pixel information [1]. Later, multi-variate and Gaussian mixture models are also introduced to model images with color information [2, 3].

Using Gaussian models is extremely computationally efficient, however the accuracy of these methods highly depends on the underlying data distribution. In other words, if the image data does not follow Gaussian distribution or something close to it, the likelihood maps will not be accurate. To overcome this problem, a random forest algorithm may be used in the proposed method. However, some aspects of the invention may use any kind of probability map generation in combination with the proposed refinements of probability maps described herein.

Random Forests

A random forest (RF) is a collection of several decision trees, and sometimes also referred to as decision forests. In general, any suitable decision tree learning model may be used, including bootstrap aggregated models (also known as bagged models) and random forest models.

Random forests are mainly used for data classification purposes in a supervised environment, which means a random forest requires labelled data during the training stage. The training stage involves learning the distributions of the data using labels for future predictions when new data arrives. Once the training is over, the same trees are used to classify new incoming samples along with the classification probabilities.

In the proposed method, the labelled information consists of the foreground and background user-input seed points. For better accuracy, the incoming image may optionally be augmented with additional information such as texture, horizontal and vertical gradient information to improve the segmentation.

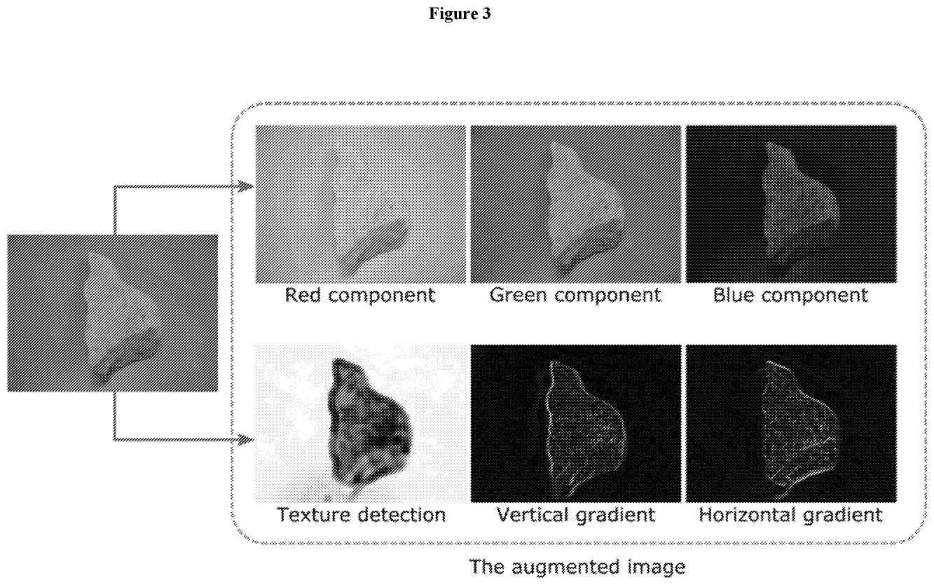

FIG. 3 provides an illustration of one procedure of constructing an augmented image 104 (FIG. 1) from the input. In the example of FIG. 3, an RGB image is taken as an input where the red, green and blue channels are analogous to multiple energy channels in multi-energy CT images. Computing gradient information is straight forward, with the average grayscale image of all the energy channels used for this purpose. However, there are several ways to approach texture synthesis [17], [18], [19]. The framework developed in [17] may be used for our purpose as it considers data from multiple channels to compute a single texture descriptor. We describe the basics of this framework below.

Texture features based on semi-local image information: It is impossible to quantify texture using information from a single pixel, since it is semi-local by nature. Semi-local information consists of a close neighbourhood around the current pixel whereas local information includes just the position and value of the pixel in consideration. This can also be called a patch.

Consider for instance a 2D grayscale image I={Iᵢⱼ:(i, j)∈Ω}. It can be viewed as a function I(x, y): Ω→R.

To denote the semi-local information, a square patch P_{xy}(I) of size n×n (an odd positive integer) centered at pixel (x, y) may be extracted.

$${𝒫}_{xy} ( I ) = \{ I ( x + k , y + l ) : k , l = - \frac{( n - 1 )}{2} , … , \frac{( n + 1 )}{2} \} \qquad ( 2 )$$

It may be considered that the given input image is obtained by discretizing a differentiable surface that allows the use of some efficient tools from classic differential geometry.

Sochen et al. [21] proposed to represent the image as Remannian manifolds embedded in a higher dimensional space. For example, a 2D gray image I:R²→R, can be viewed as a surface (denoted by Σ) with local coordinates (x, y) embedded in R³ by a mapping M_{xy}(I)→(x, y, I(x, y)).

Using formula (2) above, this manifold based representation can be extended to support semi-local information at location (x, y)

[symbol]

_{xy}(I)→(x,y,P_{xy}(I)) (3)

In the above mapping, the first two components indicate local information and the semi-local information is included in the form of a patch P_{xy}(I). From the theory of differential geometry, the area of an element on the surface M_{xy}(I) is defined as

$$‖ \frac{\partial ℳ}{\partial x} \times \frac{\partial ℳ}{\partial y} ‖$$

$$Where \frac{\partial ℳ}{\partial x} = ( 1 , 0 , \frac{\partial 𝒫}{\partial x} ) and \frac{\partial ℳ}{\partial y} = ( 0 , 1 , \frac{\partial 𝒫}{\partial y} )$$

The partial derivatives ∂P/∂x and ∂P/∂y can be computed using forward differences since the image is discrete. Using this area definition, the rate of change of the area of a surface element is defined as

$$\begin{matrix}G = ‖ \frac{\partial ℳ}{\partial x} \times \frac{\partial ℳ}{\partial y} ‖ \\ = \sqrt{( 1 + {( \frac{\partial 𝒫}{\partial x} )}^{2} ) ( 1 + {( \frac{\partial 𝒫}{\partial y} )}^{2} ) - {( \frac{\partial 𝒫}{\partial x} \cdot \frac{\partial 𝒫}{\partial y} )}^{2}}\end{matrix} \qquad ( 4 )$$

In the regions where texture is present, the intensity variations in the local neighborhood cause the corresponding surface to change with different G values. For images with more than one channel, (4) can be extended to support multiple channels. Let I=(I₁, I₂, . . . , I_{d}), where d represents the number of channels. From formula (3), the corresponding manifold based representation can be modified as M_{y}(I)→(x, y, P_{xy}(I₁), . . . , P_{xy}(I_{d})). The final rate of area change for a multi-channel image becomes:

$$G = \sqrt{( 1 + {\sum }_{i = 1}^{d} {( \frac{\partial 𝒫 ( {I}_{i} )}{\partial x} )}^{2} ) ( 1 + {\sum }_{i = 1}^{d} {( \frac{\partial 𝒫 ( {I}_{i} )}{\partial y} )}^{2} )} - {\sum }_{i = 1}^{d} {( \frac{\partial 𝒫}{\partial x} \cdot \frac{\partial 𝒫}{\partial y} )}^{2} \qquad ( 5 )$$

The texture descriptor T is finally defined as T=exp(−G²) where the exponential term applies some Gaussian smoothing. After applying the texture descriptor T to the input image I, the resulting single channel image can be denoted by Iₜ={tᵢⱼ:(i, j)∈Ω}. In a given textured region, pixel T values within that region will be similar. Across the image there may be different texture regions, where the value of T may change. In addition to the Gaussian smoothing, the semi-local nature of this texture descriptor over multiple channels makes it more robust to the noise compared to other texture descriptors [19].

While one example of use of augmentation has been described, the skilled reader will understand that other methods of image augmentation may be suitable. In particular, methods that augment the CT data in order to improve segmentation may be used. Augmentation with texture information may be used. Augmentation with horizontal and/or vertical gradient information may be used. The probability maps may be based on the augmented image data or any suitable combination of the original image data and augmented image data. In further embodiments, image data may be augmented using distance metrics (including e.g. Euclidean and/or geodesic distance metrics) at this stage, before generation of the probability maps.

Using the training information generated, the random forest algorithm generates probability maps P_{F} and P_{B} corresponding to the both foreground and background respectively as shown in FIG. 4A.

In traditional random forests, every time the user refines the segmentation output, the entire forest needs to be constructed. Whereas online random forests update their trees with the new information during each iteration, which makes them efficient. Traditional random forests are used in the disclosed method as they have shown the best results, however online random forests may also be used as part of the disclosed method.

FIG. 4B shows the probability maps generated for the foreground for two iterations. It was observed that the performance of online random forests in the second iteration is not up to the level of traditional random forests.

Probability Map Refinement Using Geodesic Distance Cues

In the proposed method, random forests are used to model the probability distribution maps based on the visual properties of a scribble or other user input over the top of an image.

The location of the scribbles also provides information about the possible location of the object.

This section aims to utilise this information to refine the probability maps [symbol] and P_{B} by computing geodesic distance maps.

The user input seed points corresponding to the foreground and background are represented as

S={ [symbol] ,S_{B}}

To refine the probability maps [symbol] and P_{B}, geodesic distances are computed from every pixel x to the seed points S_{F} and S_{B} separately to create geodesic distance maps G_{F} and G_{B}.

The geodesic distance d(x) is defined as the smallest integral of a weight function W over all possible paths from the scribbles to x. Specifically, for every x, the geodesic distance for the foreground or the background may be computed as

$${𝒢}_{l} ( x ) = {min}_{s ∈ {S}_{l}} d ( s , x ) . l ∈ \{ ℱ , ℬ \} \qquad ( 4.1 )$$

where

$$d ( {s}_{1} , {s}_{2} ) = {min}_{{C}_{{s}_{1} , {s}_{2}}} {\int }_{{s}_{1}}^{{s}_{2}} \cdot W ( x ) . {\dot{C}}_{{s}_{1} , {s}_{2}} ( x ) \vert \qquad ( 4.2 )$$

where Cs₁, s₂ indicates an arbitrary path that connects the pixels s₁ and s₂. In this way, geodesic distance maps may be constructed for both foreground and background.

The weights W, upon which the shortest paths are computed can be modelled in several ways [4, 5]. For example, in the present case, the gradients of the probability that a pixel belongs to the foreground (respectively background) are selected as the weights, i.e.

W(x)=∇ [symbol] (X)

In this case, the user provided scribbles or other user inputs are used both in generating the probability maps, and their locations are also used to improve or refine the probability maps.

In a more practical setting, images may be discretized into pixels. Assuming that each pixel is represented as a node in a graph, and has 4 neighbourhood connectivity, geodesic distance defined in equation 4.2 can be approximated as

$$d ( {s}_{1} , {s}_{2} ) = {min}_{{C}_{{s}_{1} , {s}_{2}}} {\sum }_{{x}_{1} , {x}_{2}} {W}_{{x}_{1} , {x}_{2}} \qquad ( 4.3 )$$

where

Wₓ_{₁}_{,x}_{₂}=| [symbol] (x₁)− [symbol] (x₂),x₁,x₂∈Cₛ_{₁}_{,s}_{₂}

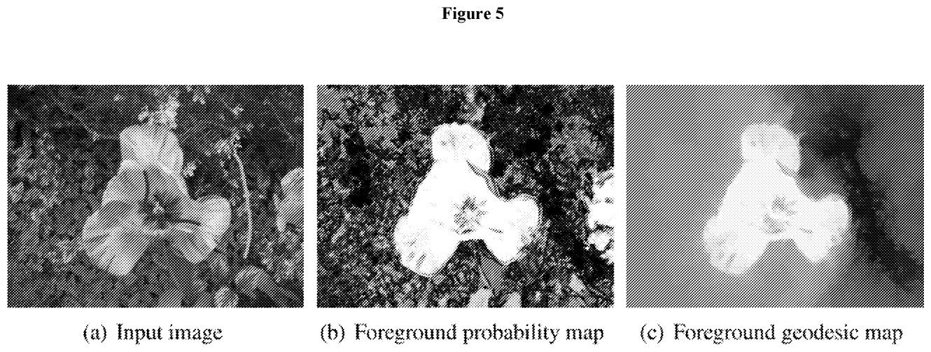

The same procedure can be repeated to compute geodesic distance maps for the background. Based on the definition of geodesic distance, a pixel x in G_{F} or G_{B} is close to the scribbles S_{F} and S_{B} if there exists a path along which the probability function doesn't change much. An example can be seen in FIG. 5. For demonstration purposes, only foreground probability maps are shown here. Note that G_{F} and G_{B} are normalized, so that the sum of probabilities at any pixel location is equal to 1.

In the foreground probability map of FIG. 5, there are some regions with high confidence other than the intended foreground object (flower at the center), such as the second flower on the right edge of the image or some leaves. This is due to their similar visual properties. In the corresponding geodesic map, it can be seen that the unwanted regions are suppressed to get a more refined confidence map.

Direct implementation of equation 4.3 involves the computation of all possible paths between any two nodes. It has a computational complexity of O(N!) and is almost impossible to compute. Yatziv et al. [6] proposed a method to efficiently compute distances in a linear time based on the special properties that grid points hold in a maintained priority queue. In this approach, geodesic distance map computation is modelled as a set of non-linear Eikonal equations solved using Fast Marching Methods [7].

Probability Map Refinement Using Euclidean Distance Cues

Euclidean distance cues from the user scribbles may also be used to refine the probability maps P_{F} and P_{B} to generate Euclidean maps ε_{F} and ε_{B}. Unlike geodesic maps, Euclidean maps do not depend on the probability maps while computing them. This property makes sure that the location information is utilized even in the cases where P_{F} and P_{B} are poorly generated. Every pixel x in the Euclidean maps ε_{F} and ε_{B} represents the likelihood that the pixel x belongs to the foreground or the background using the location information alone.

To create ε_{F} and ε_{B}, distance masks are created for both the foreground and the background. Each mask includes a set of pixels that are close to either S_{F} or S_{B}. The foreground distance mask can be computed as:

[symbol]

_{dMask}={x:d_{E}(x, [symbol] )<d_{E}(x,S_{B})} (4.4)

where

$${d}_{E} ( x , S ) = {min}_{y ∈ S} d ( x , y )$$

d(x,y) is defined as Euclidean distance between the pixels x and y. Using the mask information

$${ℰ}_{ℱ} ( x ) = 1 , ∀ x ∈ {ℱ}_{d Mask} \qquad ( 4.5 )$$

$${ℰ}_{ℱ} ( x ) = exp ( - \frac{{d}_{E} ( x , {ℱ}_{dMask} )}{{α}_{s} {σ}_{spatial}^{2} ( x )} ) , ∀ x ∉ {ℱ}_{dMask} \qquad ( 4.6 )$$

Equation 4.5 and 4.6 shows how Euclidean distance map can be calculated for the foreground. In ε_{F} the pixels within the foreground mask are given a likelihood of 1, whereas the likelihood of the pixels outside the mask varies depending on their distance from the mask, and

σₛₚₐₜᵢₐₗ(x), σₛₚₐₜᵢₐₗ(x) is a spatially varying quantity that changes its value depending on the distance from x to the foreground seed points S_{F}. It is defined as follows

σₛₚₐₜᵢₐₗ(x)=d_{E}( [symbol] , [symbol] ) (4.7)

where

$${x}_{ℱ} = arg {min}_{x} {d}_{E} ( x , {ℱ}_{dMask} ) \qquad ( 4.8 )$$

The same procedure can be repeated for the background to get a corresponding Euclidean distance map ε_{B}.

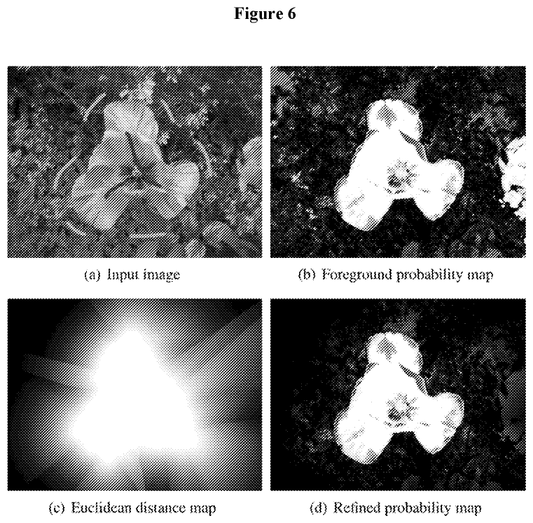

FIG. 6 shows the original probability map with user scribbles and the corresponding Euclidean distance map generated for the foreground. It can be seen in FIG. 6(c) that the pixels that belong to F_{dMask} have a constant likelihood. According to the Euclidean maps alone, the objects in this region are sure to belong to the foreground. The refined probability map in FIG. 6(d) shows that the objects far away from the intended foreground are considerably suppressed. as in equation 4.6 controls the weight given to the pixel x between its distance from the mask and user scribbles. Currently the value is set at 1.25 throughout the description, but other values may be used.

Probability Map Refinement Using Geodesic and Euclidean Distance Metrics

Geodesic and Euclidean metrics may be used to refine the probability maps in different ways: the geodesic distance metric uses image content along with the user scribble location whereas the Euclidean metric uses only location information. Here, the two are combined to generate a refined probability map. For example, the final probability of a pixel at location x is computed as

[symbol]

ₗ(x)=εₗ(x)·Pₗ(x)+u(x)· [symbol] ₗ(x),l∈{ [symbol] ,B} (4.9)

where u(x) is a tuning factor that changes according the pixel location. The main function of this adaptive tuning factor is to change the confidence of the geodesic map wherever it is necessary. In the proposed approach, it is defined as

u(x)=exp(−λ_{g}(1−| [symbol] (x)− [symbol] _{B}(x)|)) (4.10)

where, λ_{g} is empirically selected to be 2.5. The same value is used throughout the description, but other values may be used. When u(x) is close to 1, the algorithm is certain that the pixel x is in the object's interior or exterior. At locations where the algorithm is not confident, the value u(x) reaches close to 0, thereby giving less weight to geodesic maps.

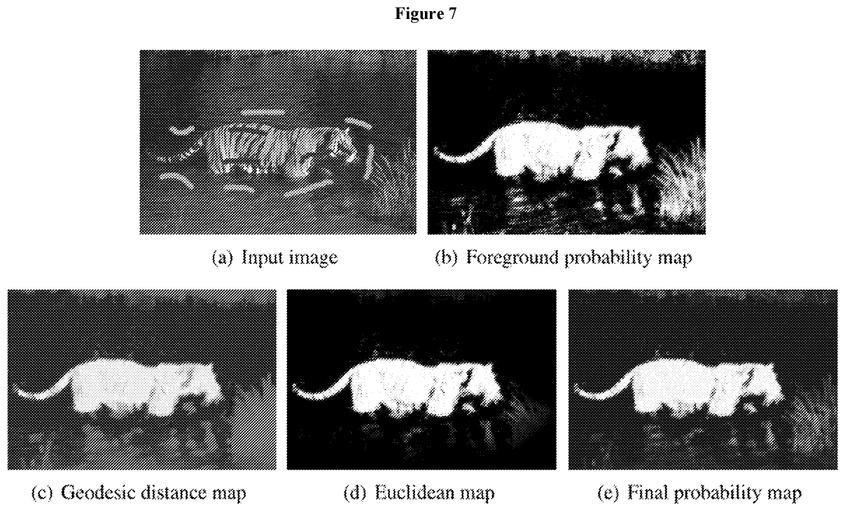

FIG. 7 shows different stages of probability map generation, starting with user scribbles to calculate the final probability map. In FIG. 7(b), the initial probability map generated using RFs has higher confidence levels for the objects (e.g. the plants at bottom right and at the top of the image) other than the intended object due to similar visual properties. However, it can be seen that the Euclidean distance maps at FIG. 7(d) and geodesic distance maps at FIG. 7(c) improved the likelihood of the foreground object using the user scribble location. The use of Euclidean distance metrics alone performed well in suppressing the false positives away from the foreground objects. The use of geodesic distance metrics helped in giving smooth probability maps and reducing false positives closer to the foreground object.

Segmentation

In the probability maps generated using RFs, the probabilities calculated for each pixel are obtained independently. Although refinement using geodesic maps enforces spatial consistency to some extent, it may not enough to get accurate segmentation. This may lead the results to be spatially inconsistent, noise sensitive, and to have rough object boundaries.

To address these problems and infer class labels for all the pixels in the input image I, a two stage CRF model is proposed for global spatial regularization. It makes use of the refined probabilities Rₗ(x) which can be reinterpreted as posterior probabilities R(l|x) to compute the final segmentation result.

Other segmentation methods such as single stage CRFs, plain thresholding, or segmentation based on Bayesian networks or Markov random fields, may be suitable in some applications. However, in general, preferred embodiments take into account neighbourhood information in the segmentation. Preferred embodiments may also use a segmentation that is solved via an energy minimization or maximum likelihood method based on probability maps.

Stage One CRF Model

In this stage, a graph is constructed from the input image I where each pixel acts like a node and has connections with 4 neighbouring pixels. The label set (l=(lᵢ)) is determined by minimizing the following energy function of the form

$$E ( l ) = {\underbrace{{\sum }_{{x}_{i} ∈ I} {ψ}_{{l}_{i}} ( {x}_{i} ) + λ}}_{region term} {\underbrace{{\sum }_{( {x}_{i} , {x}_{j} )} B ( {x}_{i} , {x}_{j} ) δ ( {l}_{i} , {l}_{j} )}}_{boundary term} \qquad ( 4.11 )$$

$${ψ}_{{l}_{i}} ( {x}_{i} ) = - log ℛ ( {l}_{i} \vert {x}_{i} ) \qquad ( 4.12 )$$

$$δ ( {l}_{i} , {l}_{j} ) = \{ \begin{matrix}1 : & {l}_{i} \neq {l}_{j} \\ 0 : & {l}_{i} = {l}_{j}\end{matrix} \qquad ( 4.13 )$$

where λ is a positive coefficient to adjust the relative importance between the regional term and the boundary term. The unary term represented by ψlᵢ(xᵢ) measures the cost of assigning a class label lᵢ to pixel xᵢ. It is computed using the information from the refined probability maps. Apart from this information, several hard constraints are imposed on the regional term at the seed pixel locations.

$${ψ}_{{l}_{i}} ( {x}_{i} ) = \{ \begin{matrix}∞ : {x}_{i} ∈ {S}_{\tilde{l}} \\ 0 : otherwise\end{matrix} \qquad ( 4.14 )$$

where l̅ is the label opposite to l. These hard constraints are set to make sure the seeded pixels are surely classified into the classes assigned by the user.

In Equation 4.11, the pairwise/boundary term is modelled as a contrast sensitive Potts model. N represents the set of all the possible unordered pairs of neighbouring pixels. B(xᵢ, xⱼ) is used to measure the penalty for a discontinuity between xᵢ and xⱼ. In this energy minimization framework, B(xᵢ, xⱼ) must be penalized heavily when xᵢ and xⱼ are similar, and use less penalty when they are very different. The property can be imitated by using several mathematical functions as shown by Veksler and Zabih [8]. However, this description uses a typical definition of B(xᵢ, xⱼ) proposed in Boykov and Jolly [9].

$$B ( {x}_{i} , {x}_{j} ) = \frac{1}{dist ( {x}_{i} , {x}_{j} )} \cdot exp ( - \frac{{( {I}_{{x}_{i}} - {I}_{{x}_{j}} )}^{2}}{2 {σ}^{2}} ) \qquad ( 4.15 )$$

where Iₓᵢ and Iₓⱼ$ represents the gray scale intensities at the locations xᵢ and xⱼ respectively. dist xᵢ and xⱼ is defined as the spatial distance between pixels xᵢ and xⱼ, and σ controls the sensitivity of the intensity differences. In this description a is set to 6. The energy minimization in Equation 4.11 is carried out by the alpha expansion algorithm proposed by Boykov et al. [10] to label individual pixels. Alternatively, the energy minimization of Equation 4.11 may be carried out by another suitable method, e.g. Gradient descent, Newton approaches, or other method. This result is then passed on to the next stage.

Stage Two CRF Model

At the end of the first stage, the resulting segmentation output has pixels either belonging to the foreground or the background. However, the one stage CRF model tends to produce geometric artefacts primarily due to the neighbourhood configuration [11, 12]. Having a dense neighbourhood is expected to lead to smoother results compared to a sparse neighbourhood, which produces geometric defects such as jagged edges and isolated patches. Unfortunately, direct increase in the neighbourhood size increases the memory consumption and computation time almost linearly.

In an interactive segmentation workflow, having larger computation times is undesirable. To address these issues, Danek et al. [13] proposed a boundary smoothing method while approximating Chan-Vese model [14] using graph cuts. A similar method may be used in the interactive segmentation context to get better results without compromising on memory or computation times.

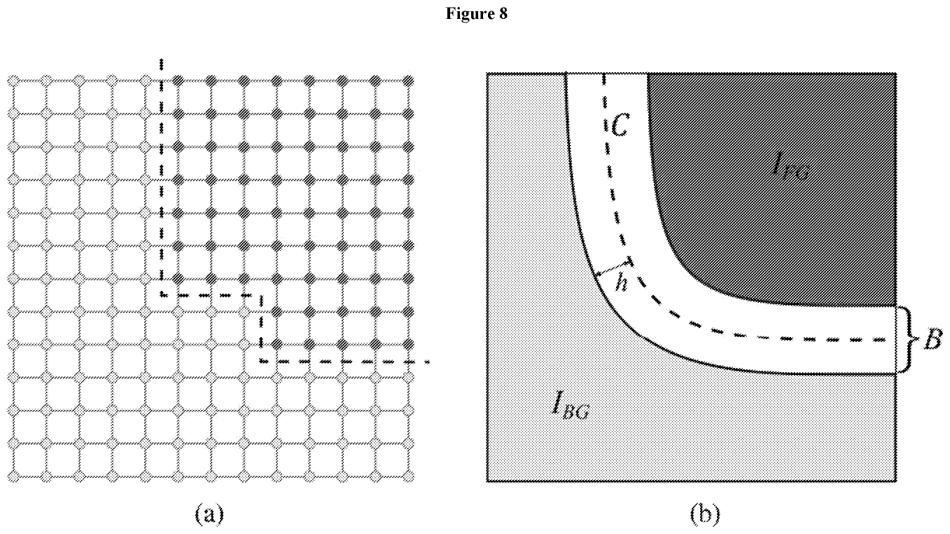

FIG. 8 (a) shows a model example of the output generated at the end of the first stage CRF model with 4 neighbour connectivity. The pixels within the regions marked in different shades of grey are classified into foreground I_{FG} and background I_{BG} respectively. The boundary between I_{FG} and I_{BG} is sometimes called the cut C. In the second stage, the goal is to improve the boundary or cut C, so it is smoother and closer to the actual object boundaries.

As pointed out earlier, denser neighbourhood graphs improve the segmentation result. In the disclosed method, a dense graph is created using only the pixels within a band B around the cut C instead of using the entire image. The band B is a set of pixels that fall within a distance or radius h from the cut as can be seen in FIG. 8(b). This significantly reduces the number of nodes to process there by having less impact on both computational and memory overhead.

Once the graph is created, the same approach followed in the previous stage is repeated to get the final segmentation output. Similarly, the unary term ψlᵢ(xᵢ) for each node/pixel is computed using the refined probability maps, and the boundary term B(xᵢ, xⱼ) is computed using the gray scale intensities. In the proposed method, the denser graph in stage two may be created using a 24 neighbourhood system, though other densities of neighbour connection may be used. To ensure the label consistency along the borders of the band B, several hard constraints are imposed using Equation 4.14. In other words, the pixels within the band whose neighbours fall in either I_{FG} or I_{BG} region are constrained to have the same label as that region.

Segmentation Performance

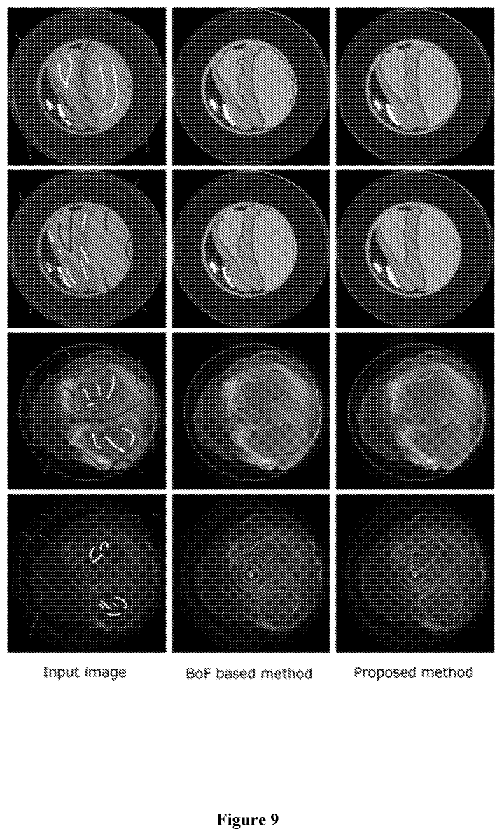

A number of experiments have been carried out on the proposed method to measure the relative performance of the method compared to a Bag of Features (BoF) method (also developed by the Applicant) and other traditional interactive segmentation algorithms such as Random Walk (RW) segmentation [15], Graph Cuts (GC) [1], Geodesic Star Convexity (GSC) prior for segmentation [5], Geodesic Graph Cuts(GGC) [4], Grabcut [3], Maximal Similarity based Region Merging (MSRM) [16]. The graphical results are only shown for the proposed method and BoF method, however performance of all the methods is tabulated. For a fair comparison, the same datasets are used here with the same input for each method. FIG. 9 shows output of the proposed method for some selected slices from a lamb chop (from now on referred to as the “lamb dataset”) (rows 1 and 2) and a knee doped with Hexabrix (from now on referred to as the “knee dataset”) (rows 3 and 4). Similarly, white and grey colour mouse scribbles are used to represent the foreground and the background respectively. The segmentation output and manually annotated ground truth are shown as grey lines.

Visual comparison of the results shows that the proposed method may provide smoother boundaries compared to the BoF method while adhering to true object boundaries. In this example, the results generated by BoF show slight block-like appearance at some places as a result of a superpixel generation pre-processing step. Since the proposed approach does not involve this step, the object boundary transition is comparatively smooth.

In the lamb dataset, the visual properties of the perspex tube in which the sample is kept are very similar to the water-like and lipid-like material. As shown in FIG. 9, the proposed approach is able to distinguish between them to some extent. Similarly, in the knee dataset, the object boundaries generated by the proposed method are closer to the true boundaries than the BoF based method. In the 4th row of FIG. 9, where the input image has limited contrast, the proposed approach shows results closer to the ground truth irrespective of the ring artefacts. Also note that the results generated here for the proposed spatially-aware segmentation approach are shown without any additional post processing steps. Post processing may involve discarding isolated regions without strokes, hole filling etc.

TABLE 4.1
Quantitative comparison of the proposed spatial-aware segmentation
approach with the other segmentation methods.

| Method | Accuracy(%) | F1 score(%) | IoU(%) |
|---|---|---|---|
| GC | 95.10 ± 2.69 | 78.53 ± 12.73 | 66.29 ± 16.63 |
| RW | 96.37 ± 1.91 | 83.19 ± 7.37 | 71.86 ± 10.67 |
| GSC | 94.13 ± 1.48 | 73.98 ± 10.66 | 59.73 ± 13.35 |
| Grabcut | 88.07 ± 1.88 | 62.90 ± 6.11 | 46.14 ± 6.56 |
| MSRM | 90.52 ± 2.32 | 63.38 ± 10.84 | 51.41 ± 12.74 |
| GGC | 94.31 ± 1.47 | 74.49 ± 10.54 | 60.35 ± 13.26 |
| BoF method | 97.91 ± 0.82 | 89.07 ± 5.08 | 80.63 ± 7.92 |
| Proposed | 98.41 ± 0.54 | 90.36 ± 7.73 | 82.64 ± 8.09 |

Table 4.1 shows the performance of the proposed approach for quantitative evaluation compared against the traditional interactive segmentation methods. From the results, it is clear that the proposed method has superior performance than all the other methods. Although, BoF method has performance close to the proposed method with low standard deviation over the results.

However, the difference in the performance metrics is not as evident as in the visual comparison of the results. This may be because the metrics chosen here only quantify the average deviation between the given result and the ground truth images.

Effect of Distance Cues on Segmentation

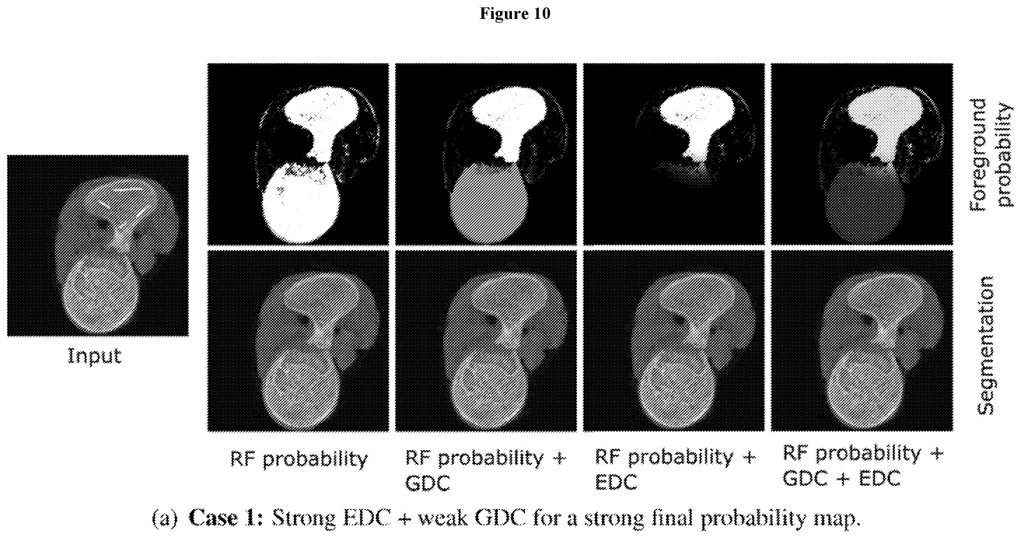

One of the advantages of the proposed spatially-aware segmentation is that it fine tunes the probability maps using various types of spatial cues. In this disclosure, three cases (FIGS. 10-12) are presented to show the advantages of incorporating these spatial metrics or cues (Euclidean and geodesic) in the method. In each case, the operating conditions were set to either support or oppose Euclidean distance cues (EDC) and/or geodesic distance cues (GDC). In all cases, it was visually shown that irrespective of the intermediate probability maps, the final probability maps resulted in segmentation closer to the ground truth. The intermediate probability maps include probability maps generated by random forest (RF probability), RF probability+GDC, RF probability+EDC, and the final probability map (RF probability+GDC+EDC). For the purpose of testing, three different datasets were used for the demonstration.

The first dataset was a multi-energy CT scan of a sheep femur (sheep femur dataset). The volume dimensions are 672×672×384 voxels, the cubic voxels are 0.090 mm wide, and consists of 4 attenuation channels. The task in this dataset was to segment the upper portion of the femur bone.

For the second dataset, a publicly available human CT dataset is used. This dataset dimensions are 512×512×123 voxels with a voxel size of 0.734×0.734×3.20 mm³. This dataset also has the annotations for the liver available.

Finally, the third dataset was of a tibial plateau series. The volume dimensions are 512×512×160 voxels, the cubic voxels are 0.10 mm, and consists of 5 attenuation channels. In this dataset, the bone region was to be segmented from the gadolinium enhanced cartilage.

Case 1: The first case demonstrates the effect of a strong EDC and weak GDC on the proposed method. As part of the experiment, the user scribbles (dark—background, white—foreground) are given to segment upper portion of the bone as shown in FIG. 10. The top row shows the probability maps and the bottom row shows the corresponding segmentation results. It can be observed that the entire bone region was given a high foreground probability due to similar visual properties and failed to utilize the location information provided by the user strokes. However, the intermediate probability refinement steps using EDC and GDC tend to suppress the lower bone region.

It can be seen that the GDC based probability refinement still highlights the lower bone portion. This is because of the way geodesic distances are calculated, which takes into account the difference in the neighbouring pixels' likelihoods. A small gap in between the upper and lower bone regions allows the geodesic paths to pass through with higher confidence. Hence, the lower bone region still has high foreground probability and is subsequently segmented as a foreground. On the other hand, EDC doesn't take into account the values at pixel locations, so it was able to suppress most of the unwanted bone region.

Therefore, the segmentation result using EDC was closer to the ground truth. The final probability map, which is an adaptive combination of both Euclidean and geodesic distance maps gives segmentation close to the ground truth irrespective of weak geodesic maps.

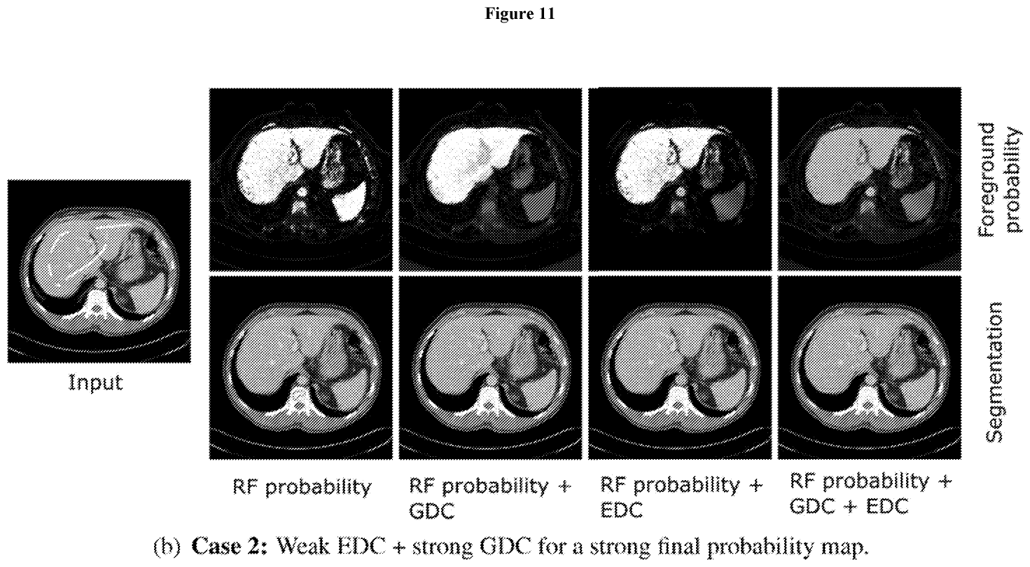

Case 2: The second case as shown in FIG. 11 presents the effect of a strong GDC and weak EDC on the proposed method. The user scribbles are provided to segment liver from a human CT dataset. As with case 1, just using the probability maps generated using random forests resulted in an inaccurate segmentation with a lot of false positives as shown in FIG. 11. In the further stages of refinement, when the EDC was applied, the resulting segmentation has false positives as well as some false negatives within the liver. However, since GDC incorporates pixel values, the segmentation result was smooth and accurate to the ground truth. The combination of these two also resulted in a segmentation closer to the ground truth without any false positive or false negative patches.

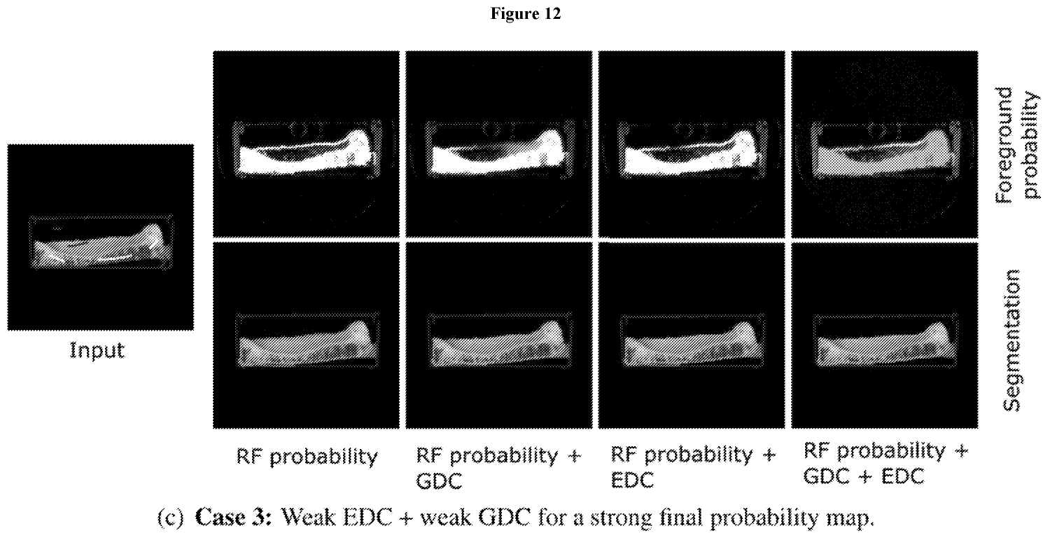

Case 3: The third scenario shows that the final probability map can yield better segmentation results even in the cases where Euclidean and geodesic distance cues fail. As it can be seen in FIG. 12, bone segmentation using random forest based probabilities alone over-segmented the bone region and falsely included the surface region of the cartilage. The refinement using GDC and EDC have correspondingly resulted in either over-smoothing or have a slight change in the original probability map. This can be observed in their segmentation results. However, in the final probability map, the optimal combination of both Euclidean and geodesic maps has shown a better trade-off between the foreground and the background probabilities, which in turn resulted in an accurate segmentation. Refinement of the probability map using a combination of Euclidean and geodesic distance metrics or cues therefore provides a significantly improved result over the use of either Euclidean or geodesic distance metrics or cues alone.

Accuracy Evaluation Using Two Stage CRF Model

As described above, a two stage CRF model was deployed as a final step to get better segmentation results in terms of accuracy and boundary smoothness. A number of validation experiments were performed, and the results visually show how a two stage CRF benefits the segmentation.

For the purpose of validation, two datasets were used to compare stage one results against stage two. The first dataset was a knee dataset. As before, the task was to segment the bone from this dataset. For the second dataset, a scan of a sheep femur was used (from now on referred to as the “sheep femur dataset”). The volume dimensions are 480×480×384 voxels, the cubic voxels are 0.090 mm wide, and consists of a single material channel (calcium). In the sheep femur dataset, there were three classes: cortical bone, trabecular bone, and the background. In the following test, the experiments demonstrate the case where trabecular bone was considered foreground and the rest as background. Trabecular bone is a porous structure within a thick bone shell. One of the main reasons to choose this dataset is because the trabecular bone is inherently porous and non-homogeneous in appearance.

As the main purpose of having a two stage CRF is to ensure correct boundaries, a new performance metric, mean absolute surface distance (MASD) may be introduced to quantify this property. MASD measures the average minimal distance between two boundaries corresponding to the ground truth and the segmented image. It returns the distance value in pixels, and the closer these values get to 0, the greater the resemblance between the ground truth and the corresponding segmented image.

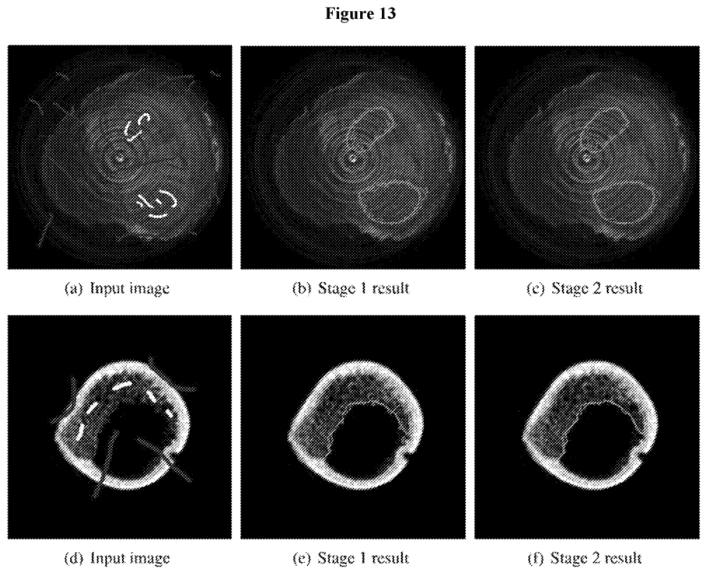

FIG. 13 shows the results for the given input after stage one and stage two of the proposed method. The user strokes are shown using white and dark strokes for the foreground and the background respectively in images (a) and (d). The ground truth is annotated using grey contours and the outcome of stage one and stage two are also shown using grey contours in the stage 1 and 2 results.

The first row in FIG. 13 is an image taken from the knee dataset. It can be observed that the improvement of segmentation boundary smoothness when using the two stage methods. Moreover, the result from stage one has holes and other falsely segmented regions. Although these regions could be removed up to some extent in post-processing using connected component analysis, this approach doesn't help in getting smooth and accurate boundaries.

A second stage of CRF model is different because it takes the image content into consideration while refining the borders. Similarly, in the second row of FIG. 13, stage one and two of the trabecular bone segmentation in the sheep femur dataset is illustrated. The non-homogeneous nature of the trabecular region led to rough surfaces and under segmentation of the trabecular bone.

Along with the visual results, Table 4.2 outlines the average performance of the proposed method at the end of stage 1 and stage 2 over these two datasets shown in FIG. 13. It can be observed that the results after stage two show better performance than the stage one. Although, the accuracy, F1 score, and IoU only show slight improvement over stage one, visual analysis shows significant difference between the results. The newly introduced metric MASD shows that introducing a second stage CRF increases the performance of the algorithm by almost 40%.

TABLE 4.2
Performance comparison of the proposed approach
after a single stage and double CRF models.

| Method | Accuracy(%) | F1 score(%) | IoU(%) | MASD(pixels) |
|---|---|---|---|---|
| Stage 1 | 98.29 ± 0.59 | 91.22 ± 6.06 | 84.34 ± 9.21 | 10.85 ± 5.45 |
| Stage 2 | 98.89 ± 0.52 | 92.86 ± 4.93 | 85.28 ± 7.67 | 6.16 ± 3.26 |

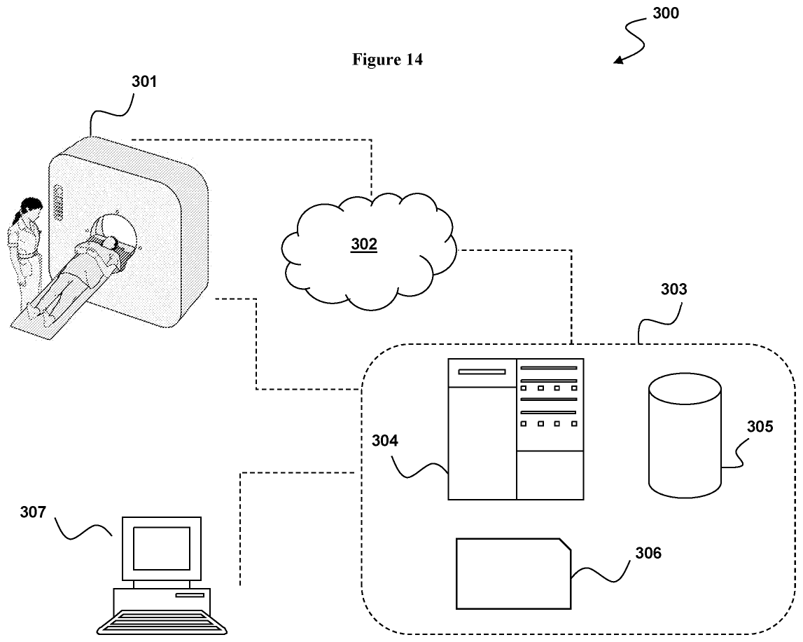

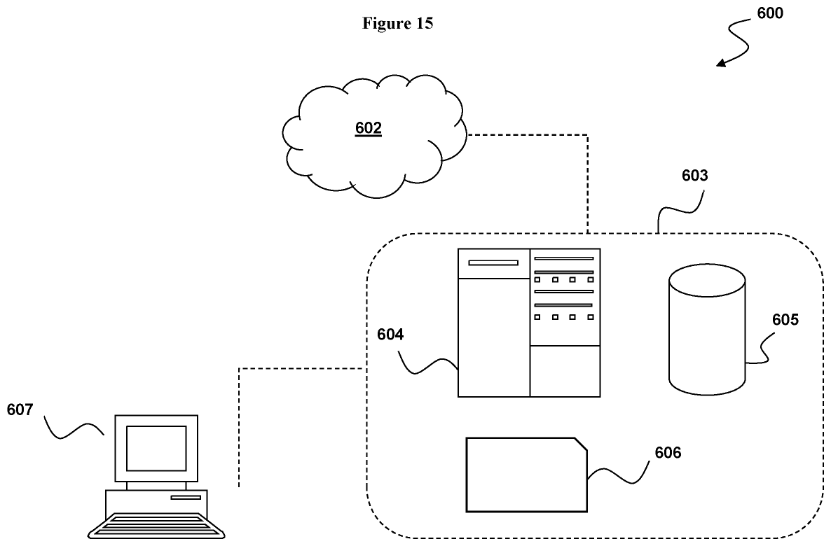

The computer implemented methods of the present invention are outlined as method 200 in FIG. 2 in one preferred embodiment and may be operated using the systems as exemplified in FIGS. 14 and 15.

In use with method 200 of FIG. 2, the system 300 of FIG. 14 includes a multi-energy CT imaging scanner 301. Multi-energy CT images are taken of a subject (e.g. an object/sample or patient of interest) 210 and the image data set delivered via communications network 302 to a user interface 307 via computer system 303. Computer system 303 includes processor 304, data storage medium 305 and server 306.

Processor 304 receives the image data set from communications network 302 and implements the processing steps of the methods of the present invention together with data storage medium 305 and user input received from interface 307. Server 306 serves and receives data between computer system 303 to user interface 307.

FIG. 15 shows an alternate system 600 of the present invention, where multi-energy CT image data is received over a communications network 602 by a processor 604 within computer system 603. Multi-energy CT data is received over communications network 602 from an external source via known communications protocols for example, email, internet, FTP or HTML.

As with system 300 computer system 603 includes processor 604, data storage medium 605 and server 606. Processor 604 receives the image data set from communications network 602 and implements the processing steps of the methods of the present invention together with data storage medium 605 and user interface 607. Server 606 serves data to and from computer system 603 to interface 607.

The various operations of methods described above may be performed by any suitable means capable of performing the operations, such as various hardware and/or software component(s), circuits, and/or module(s). Generally, any operations illustrated in the Figures may be performed by corresponding functional means capable of performing the same or equivalent operations.

The various illustrative modules and method steps described in connection with the embodiments disclosed herein may be implemented as electronic hardware, computer software, or combinations of both. To clearly illustrate this interchangeability of hardware and software, various illustrative components, modules, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the embodiments of the invention.

The steps of a method or algorithm and functions described in connection with the embodiments disclosed herein may be embodied directly in hardware, in a software module executed by a processor, or in a combination of the two. If implemented in software, the functions may be stored on or transmitted over as one or more instructions or code on a tangible, non transitory computer-readable medium.

A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD ROM, or any other form of storage medium known in the art.

A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blu ray disc where disks usually reproduce data magnetically, while discs reproduce data optically with lasers.

Combinations of the above should also be included within the scope of computer readable media. The processor and the storage medium may reside in an ASIC. The ASIC may reside in a user terminal. In the alternative, the processor and the storage medium may reside as discrete components in a user terminal.

The examples above relate primarily to segmentation into two regions (which may be considered ‘foreground’ and ‘background’ or may be given some other label). However, the invention may also extend to segmentation into more than two regions. For example, the process may be repeated with different foreground and background targets each time to ultimately yield three or more regions.

Three-Dimensional Segmentation

Further, while the invention has been described primarily in relation to a two dimensional ‘slice’ of multi-energy CT data, it may be possible to extend the method to segmentation of three dimensional data. For example, an initial segmentation on 2D data may be extended into 3D data. Alternatively, multiple segmentations may be performed in different 2D ‘slices’ before extension into 3D data.

Further, if a 2D segmentation is to be extended to 3D, the initial 2D segmentation may be an interactive segmentation based on user-input markings, strokes or similar. Segmentations of further slices or otherwise extending the 2D segmentation into 3D may be performed without further user-input of markings/strokes etc. This may be done using the labels and vectors already determined, or further strokes, markings etc. may be automatically created. The original user input may be propagated to other slices in any suitable way.

The above 2D approach may be extended for segmentation of volumetric spectral CT data. Spatial awareness may be retained across the volume with limited user intervention.

The proposed method may include:

1. Collecting the user input (e.g. mouse scribbles) on a single slice, which we will refer to as a ‘start-slice’.
2. Automatically propagating labels from the start-slice to the adjacent slices until the entire volume is segmented.
3. Applying a dense CRF along the object borders to ensure smooth boundaries across all the slices.

As an alternative to the proposed slice-wise approach, the 2D method may be extended to whole-volume segmentation. However, as discussed below, the proposed slice-wise approach is expected to provide better performance in terms of usability and/or accuracy and/or computational resource requirements.

Volumes are not as interactive as 2D images when using traditional input devices like a mouse and keyboard. However, constraining the user input to a single slice during whole-volume segmentation does not provide the full context in the volume due to the change in visual properties and the object shape across the slices. In such situations, having 3D input tools may help users handle 3D user input for volumes of data, and potentially help users provide 3D scribbles in the volume directly or 2D scribbles using arbitrary slice views. Although 3D input tools are an option, this requires additional hardware and additional user training.

A preferred approach is slice-wise with limited user input (e.g. input only in the start-slice). In this approach, a user can select any one slice (or a slice may be automatically selected) along the axis of their choice, and provide the input for segmentation.

Then, an automatic label propagation is introduced to transfer these user-defined labels across other slices. Due to the transfer of user input to every slice, the slice-wise propagation is expected to perform better at dealing with the object's appearance changes.

The user input based on a single slice may be used in refinement of 3D probability maps. However, as geodesic map computations rely on the volume content, the change in visual properties of the object across the volume may lead to a lower confidence of the foreground and background during probability map refinement. The Euclidean distance-based refinement, which works solely based on the location of the seed points, may be affected by changes in the object's shape across the volume. To overcome these problems, the proposed slice-based automatic label propagation may take into account the object context in every slice.

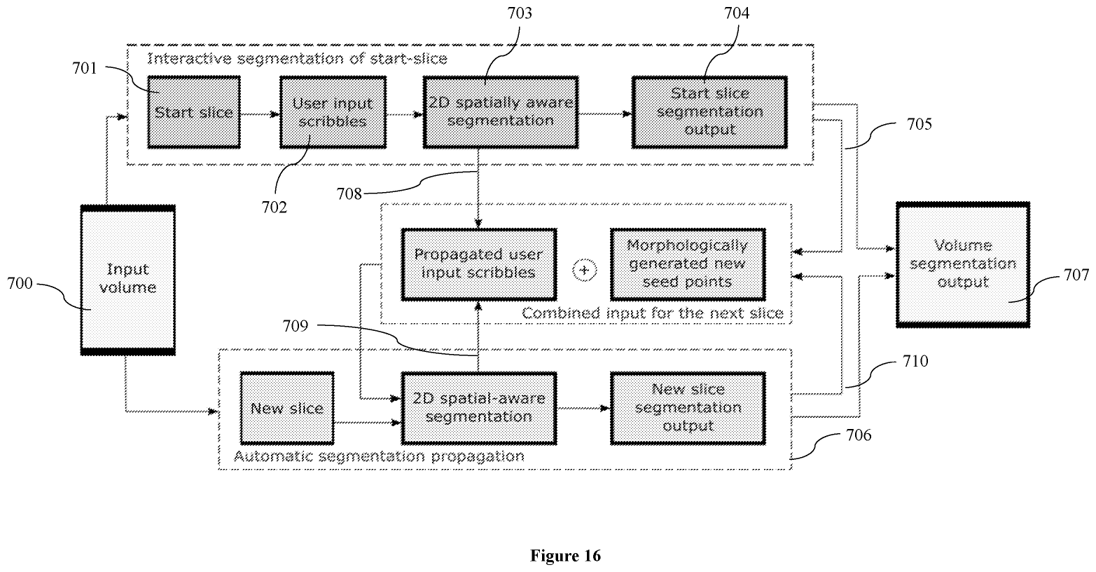

A possible workflow for a slice-wise spatially aware segmentation method for a multi-energy CT volume is illustrated in FIG. 16. The user picks a slice 701 from the volume 700 (the ‘start-slice’) and provides user input 702 (e.g. by drawing mouse scribbles) to represent the foreground and background regions. Once the user input is provided, any method described herein may be used 703 to segment the start-slice 704. The result can be further refined using more user scribbles if necessary. Once the segmentation is obtained for the start-slice, this result is automatically propagated 705 to the adjacent slices. Similarly, all the other remaining slices are sequentially segmented 706 without any additional user intervention. Once the volume is segmented 707, a dense CRF or other post-processing may be employed along the object borders to smooth out the inter slice variations in the segmentation result.

The user may select a start-slice from anywhere within the volume. However, it is convenient to select a slice approximately at the center of the region or object of interest within the volume. This helps limit the potential for errors to build up as the segmentation propagates away from the start-slice. Selecting a central slice as a start-slice also allows processing to both sides of the start-slice simultaneously to reduce computation time.

In the proposed slice-wise segmentation, the automatic propagation of segmentation from the start-slice requires the user input to be propagated 708, 709 from the start-slice to the neighbouring slices sequentially.

To propagate the user scribbles from the start-slice to the adjacent slices, an assumption may be made that the adjacent slices in a volume are similar to each other. With this assumption, during the inference stage of segmentation of slice Sᵢ, a graph may be constructed that not only includes pixels from Sᵢ, but also the pixels located adjacent to the seed points in Sᵢ₊₁. Therefore, when the segmentation is complete for Sᵢ, it will also have generated a set of seed points in the slice Sᵢ₊₁ for both the foreground and the background. The same procedure is repeated sequentially at every slice. When Sᵢ is the start-slice, the inference stage generates seed points for both the slices adjacent to it.

This way, the user-input information is propagated from one slice to the others. To make sure all the user propagated scribbles are correct, some of the new seed pixels generated for Sᵢ₊₁ may be discarded if they fall too close to the object boundaries in Sᵢ.

In addition, morphology based information may be used. In some embodiments, user input from the start-slice (or the propagated user-input in later slices) is retained and may be combined with morphological operations to generate seed points for the next slice.

In the proposed method, once the segmentation is performed for slice Sᵢ, this result is used to generate 710 new seeds for Sᵢ₊₁. Here, the assumption is that, though the segmentation result is not very accurate near object boundaries, it will have a high confidence away from the boundaries. Therefore, a morphological skeleton may be generated to get seed points for the foreground. Similarly, one or more morphological erosion operators with a given radius may be used to get a band of seed points around the object for the background.

In Wang et al. [22], which relates to segmentation of MRI data, a morphological erosion operation is applied only once, and the result is used for the new background seed points. Due to the large number of background seed points, this usually results in a bias towards the background class, and extra computational overhead. Wang et al. [22] used a dynamically balanced online random forest to overcome the bias problem. However, in contrast, the Applicant proposes to select only a band of pixels around the object. The spatial distance metrics or cues then help the segmentation result to be contained within this band.

The combination of both user-propagated scribbles and the morphology-based scribbles also reduces the label propagation dependency on the shape of the object to some extent. For example, if the segmented object in Sᵢ is a near circular shape, the morphological skeleton may extract very few foreground seeds at the center of the object, resulting in generating poor probability maps. In the proposed method, the propagated user-input scribbles along with the morphology-based scribbles provide enough seed points to support acceptable probability maps.

As shown in the FIG. 16, the seed points generated using both methods are combined to segment the slice Sᵢ₊₁. The same spatially aware segmentation method may be used for all the slices. Since the start-slice is chosen approximately at the center of the region or object of interest, the segmentation result propagates towards both ends of the targeted region. The propagation in either direction may stop if the method does not find any seed points for the current slice. At the end, the segmentation output for all the slices may be stacked together to get the final segmented volume.

In the case of a poor segmentation result in one of the propagated slices, errors may be propagated to further slices.

Further, if the object of interest is discontinuous across slices, the method may require each disjoint region to be provided with user-input seed points.

These problems may be addressed by any one or more of the following methods. Potentially problematic areas may be selected (by the user or automatically) as the start-slice to give the best input data to problematic areas. Further user input may be provided in problematic areas. Further user-input may be solicited automatically when required. Multiple start-slices may be used, with propagation in both directions to meet in between the start slices. Any combination of these methods may be used.

After propagating segmentation to all the slices, the volumetric segmentation generally results in inter-slice variations as the slices are processed independently from each other. To correct this behaviour, a dense CRF method similar to the 2D CRF method described above may be used. In this case, a small band of voxels may be chosen around the object boundary in the volume. A dense CRF model (e.g. with a system of 32 connecting neighbourhood voxels around each voxel) may be constructed. A 32-voxel neighbourhood involves all the immediate 26 neighbours in the 3×3×3 block, and the 6 center voxels located at the faces of the 5×5×5 cube around each voxel. Other neighbourhoods may be suitable. This dense CRF model may be optimized using an alpha expansion method to get a smooth volume segmentation result. Other forms of post-processing to smooth inter-slice variations may be used.

For the purpose of testing 3D segmentation, two MARS datasets and two synthetic datasets were used. The MARS data sets include:
Sheep femur dataset—A single channel dataset of a sheep femur with 377×365×60 voxels, which are all cubic with a width of 0.09 mm.
Plateau series dataset—Five attenuation channels of an excised human tibial plateau with 462×400×23 voxels, which are all cubic with a width of 0.10 mm

The synthetic datasets include:
Cylinder dataset—A 200×200×80 volume that has a cylinder with radius of 32 pixels.
Pyramid dataset—A pyramid with a square cross section within a 200×200×80 volume.

Both the synthetic datasets consist of single channel, and were corrupted with severe speckle noise to minimize the distinction between foreground and background.

Slice-Based Method Vs. Whole-Volume Segmentation

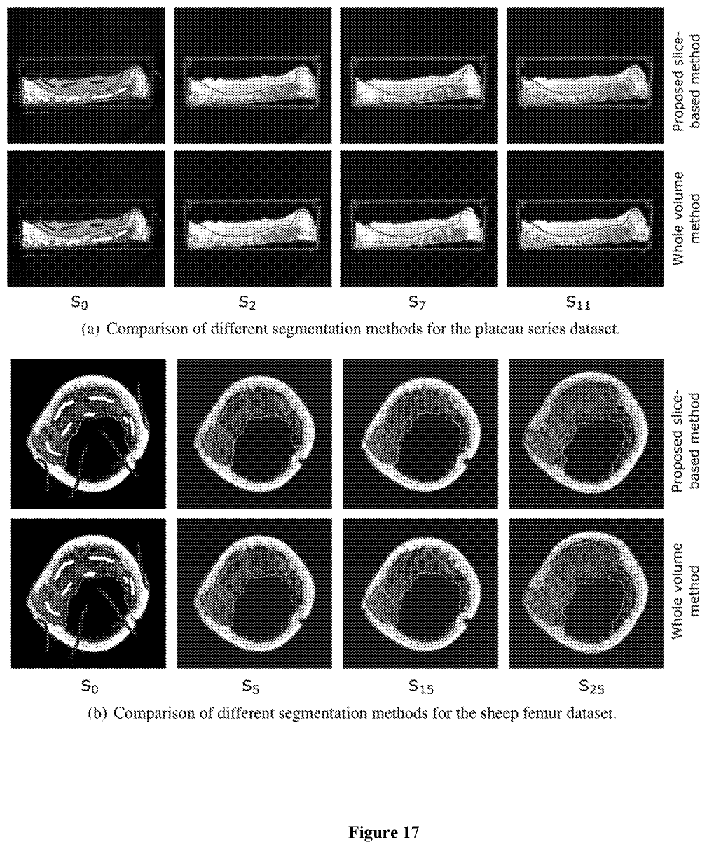

FIG. 17 shows an example of the segmentation result for two different datasets using the two methods in comparison, i.e. a slicewise method against whole-volume segmentation using the direct extension of the 2D method described above.

In FIG. 17(a), the segmentation results are shown for multiple slices from the plateau series dataset. It can be seen that the visual performance of the both methods is similar to each other. However, the proposed method was able to segment the upper edge of the object slightly closer to the ground truth across all the slices.

The segmentation of the trabecular bone in sheep femur is shown in FIG. 17(b). The visual comparison shows that the proposed slice-based method generated a smooth segmentation boundary closer to the ground truth. In the whole-volume based method, the result includes a lot of false negatives inside the object.

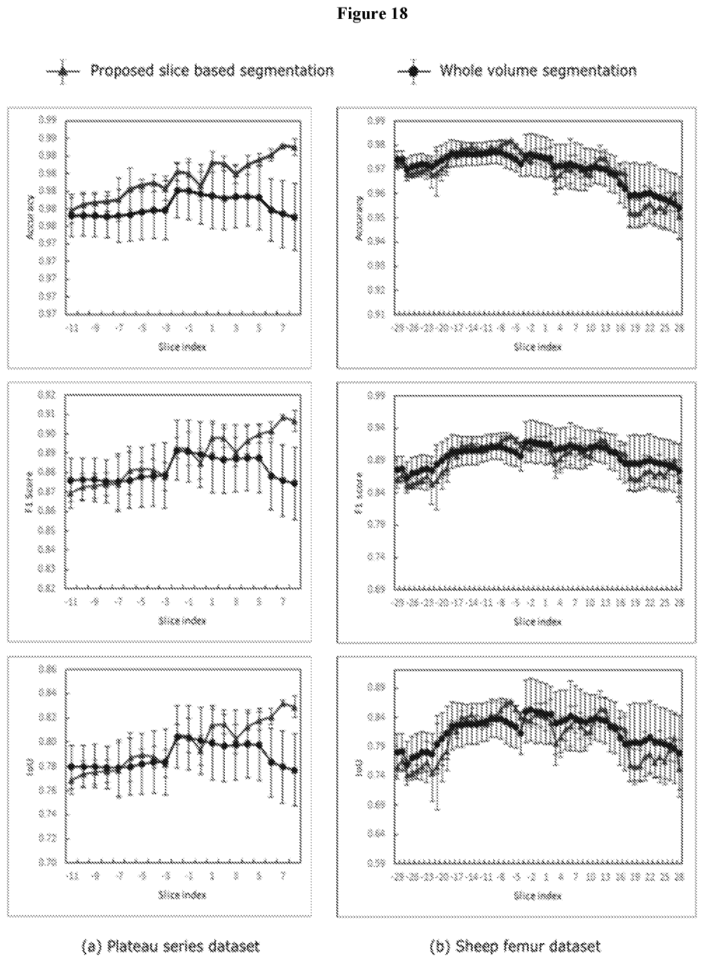

FIG. 18 shows the plots for accuracy, F1 score, and Intersection over Union (IoU) for each slice in the two MARS datasets. The plotted data is the average performance calculated after testing the data with 10 different sets of user input for specifying same foreground and background. For each slice, the error bars show the standard deviation and mean of the accuracy, F1 score and IoU. In each plot, the results are shown to compare the whole-volume based method (black) with the proposed method (grey).

For the plateau series dataset (FIG. 18(a)), the proposed slice-based method has better performance than the whole-volume method overall. Though the start-slice performance is similar for both methods, the propagation resulted in an average performance increase in the slice-based method. The low standard deviation for each slice indicates less variability between the results using different sets of user input. In the given three performance plots, the whole-volume method has a higher accuracy at the start-slice located at the center and gets worse as you move away from it.

In the sheep femur data set segmentation, the quantitative results are shown for the two segmentation methods in comparison. FIG. 18(b) shows that the average performance of both methods is similar, and fluctuates across different slices. Similar to the plateau series dataset, the low standard deviation in the measurements indicate stability over different sets of user input. In these plots, it can be seen that the performance of both the methods started to degrade as they move away from the start-slice in the center.

TABLE 5.1

| | Computation time (sec.) | Memory usage (MB) |
|---|---|---|
| | Plateau series dataset (462 × 400 × 23) | |
| Whole-volume | 24.70 | 2324 |
| segmentation | | |
| Proposed slice-based | 22.83 | 142 |
| segmentation | | |
| | Sheep femur dataset (377 × 365 × 60) | |
| Whole-volume | 36.32 | 4540 |
| segmentation | | |
| Proposed slice-based | 35.54 | 137 |
| segmentation | | |

Table 5.1 provides a quantitative comparison of the computational requirements for the whole-volume and the slice-based segmentation methods. Both the volumes used were cropped to the dimensions mentioned in the table. Table 5.1 shows the average memory usage and the time required to complete the segmentation task by both methods. The execution time for both the methods is close to each other. However, there is a large difference in the memory requirements for the two approaches. Compared to the whole-volume method, the slice-based method needs almost 16 times and 33 times less memory for the plateau series and sheep femur datasets, respectively. This is because, during the CRF stage, the graph that needs to be constructed for the whole volume is much larger and requires more connections between the nodes than the graph that is required for each slice. In the slice-based method, as this can be avoided, the memory requirement is significantly smaller. Further, these datasets are small in comparison to some datasets expected in clinical settings. The larger the datasets, the higher the impact of these memory requirements.

The Dependency of Label Propagation on Object Shape

Two synthetic datasets, the cylinder and the pyramid, were used to investigate the effect of the object's shape during segmentation. The comparison was done for a first method in which the propagated labels consisted of morphology based scribbles as well as the propagated user scribbles, against a propagation method including only morphology based scribbles. For the pyramid dataset, the visual analysis of the segmentation in the adjacent slices does not show any noticeable difference between both methods even though the proposed method propagates more foreground seed points to the adjacent slice.

For the cylinder dataset, the combination of morphological and user-input scribbles performed better. One of the reasons for this result is the shape of the object. When only the morphology based scribbles are used, the square shaped object in Si could create enough foreground seed points to propagate to Sᵢ₊₁, whereas the circular object could not satisfy this criteria. Ideally, a perfect circle only creates a single skeleton point at the center. Having fewer seed points affects the primary step of generating probability maps.

TABLE 5.2

| | Accuracy | F1 score | IoU |
|---|---|---|---|
| | Pyramid dataset | | |
| Morphology based scribbles | 0.96 ± 0.02 | 0.90 ± 0.07 | 0.83 ± 0.11 |
| Morphology based + | 0.96 ± 0.02 | 0.91 ± 0.06 | 0.85 ± 0.11 |
| propagated user scribbles | | | |
| | Cylinder dataset | | |
| Morphology based scribbles | 0.67 ± 0.19 | 0.53 ± 0.18 | 0.39 ± 0.20 |
| Morphology based + | 0.93 ± 0.01 | 0.89 ± 0.01 | 0.80 ± 0.02 |
| propagated user scribbles | | | |

Table 5.2 shows a performance comparison of label propagation methods using synthetic datasets. For the pyramid dataset, the results for both the approaches are very close to each other with slightly better performance using the combined morphology and propagated user input. However, the cylinder dataset has a large margin between the scores. This indicates that the shape of the object can have a marked influence on the segmentation propagation.

The Effect of Reduced Background Seed Points

TABLE 5.3

| | Accuracy | F1 score | IoU | Time (sec) |
|---|---|---|---|---|
| | Plateau series dataset | | | |
| Complete | 0.98 ± 0.01 | 0.87 ± 0.02 | 0.77 ± 0.03 | 7.89 ± 0.69 |
| background | | | | |
| seed points | | | | |
| Reduced | 0.98 ± 0.01 | 0.88 ± 0.01 | 0.80 ± 0.02 | 0.889 ± 0.02 |
| background | | | | |
| seed points | | | | |
| | Sheep femur dataset | | | |
| Complete | 0.89 ± 0.06 | 0.64 ± 0.26 | 0.53 ± 0.26 | 3.02 ± 0.34 |
| background | | | | |
| seed points | | | | |
| Reduced | 0.97 ± 0.01 | 0.89 ± 0.03 | 0.78 ± 0.04 | 0.849 ± 0.03 |
| background | | | | |
| seed points | | | | |

Table 5.3 shows a quantitative analysis of the segmentation performance under two different methods of background label propagation for plateau series dataset and sheep femur dataset. This test studies the effect of the morphological operators to produce seed points during propagation. As mentioned above, the method from Wang [22] produces a large number of background seed point while the preferred approach reduces the set of background seed points to a thin outline. The reported time is for processing each slice. The performance for the plateau series is relatively similar for both methods. However, during the sheep femur dataset segmentation, having a biased number of seed points resulted in a lower accuracy than its counterpart. One of the reasons for the poor probability maps using the complete set of background seed points is that the number of seed points used for the foreground was much smaller than the number of background seed points. This unbalanced ratio made the probability maps biased towards the background, which in turn resulted in reduced quality.

It can also be noted that the performance scores of the proposed reduced background seed points for both the datasets are very similar, which shows the robustness of the proposed method irrespective of the change in the dataset.

Table 5.3 also shows the per-slice computation time required for both datasets using two background seed generation approaches. For the plateau series dataset, the time required to process each slice has increased by nearly 10 times. Similarly for the sheep femur data, the processing time has increased by almost four times. This shows that having a larger number of seed points not only affects the segmentation result, but also increases the processing time significantly.

The segmented data resulting from the Applicant's process may be displayed to a user on any suitable display.

Interactive segmentation algorithms often give little attention to the spatial cues provided by the user input (e.g. mouse scribbles). The method of the example described herein may use a spatially aware method with a two stage CRF model. Once the user input is given, a random forest model uses underlying data distributions to generate probability maps corresponding to both foreground and background.

The method may use both Euclidean and geodesic distance cues to refine these probability maps to bring spatial awareness. With the help of the refined probability maps, a two stage CRF model may be used to obtain a segmentation output with smooth object boundaries. Also demonstrated above is a series of experiments to validate different steps of the proposed method. The proposed approach outperforms existing methods and gives smooth and accurate results.

While the invention has been described principally with reference to the segmentation of multi-energy CT data, other medical image data, including 3D medical image data, may also be segmented.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

### REFERENCES

[1] Boykov, Y. Y., & Jolly, M. P. (2001, July). Interactive graph cuts for optimal boundary & region segmentation of objects in ND images. In Proceedings eighth IEEE international conference on computer vision. ICCV 2001 (Vol. 1, pp. 105-112). IEEE.
[2] Dai, S., Lu, K., Dong, J., Zhang, Y., & Chen, Y. (2015). A novel approach of lung segmentation on chest CT images using graph cuts. Neurocomputing, 168, 799-807.
[3] Rother, C., Kolmogorov, V., & Blake, A. (2004). “GrabCut” interactive foreground extraction using iterated graph cuts. ACM transactions on graphics (TOG), 23(3), 309-314.
[4] Price, B. L., Morse, B., & Cohen, S. (2010, June). Geodesic graph cut for interactive image segmentation. In 2010 IEEE computer society conference on computer vision and pattern recognition (pp. 3161-3168). IEEE.
[5] Gulshan, V., Rother, C., Criminisi, A., Blake, A., & Zisserman, A. (2010, June). Geodesic star convexity for interactive image segmentation. In 2010 IEEE Computer Society Conference on Computer Vision and Pattern Recognition (pp. 3129-3136). IEEE.
[6] Yatziv, L., Bartesaghi, A., & Sapiro, G. (2006). O (N) implementation of the fast marching algorithm. Journal of computational physics, 212(2), 393-399.
[7] Sethian, J. A. (1999). Fast marching methods. SIAM review, 41(2), 199-235.
[8] Veksler, O., & Zabih, R. (1999). Efficient graph-based energy minimization methods in computer vision. New York, USA: Cornell University.
[9] Boykov, Y. Y., & Jolly, M. P. (2001, July). Interactive graph cuts for optimal boundary & region segmentation of objects in ND images. In Proceedings eighth IEEE international conference on computer vision. ICCV 2001 (Vol. 1, pp. 105-112). IEEE.
[10] Boykov, Y., Veksler, O., & Zabih, R. (2001). Fast approximate energy minimization via graph cuts. IEEE Transactions on pattern analysis and machine intelligence, 23(11), 1222-1239.
[11] Klodt, M., Schoenemann, T., Kolev, K., Schikora, M., & Cremers, D. (2008, October). An experimental comparison of discrete and continuous shape optimization methods. In European Conference on Computer Vision (pp. 332-345). Springer, Berlin, Heidelberg.
[12] Boykov, Y., & Kolmogorov, V. (2003, October). Computing geodesics and minimal surfaces via graph cuts. In null (p. 26). IEEE.
[13] Daněk, O., Matula, P., Maška, M., & Kozubek, M. (2012). Smooth Chan—Vese segmentation via graph cuts. Pattern Recognition Letters, 33(10), 1405-1410.
[14] Chan, T. F., & Vese, L. A. (2001). Active contours without edges. IEEE Transactions on image processing, 10(2), 266-277.
[15] Grady, L. (2006). Random walks for image segmentation. IEEE transactions on pattern analysis and machine intelligence, 28(11), 1768-1783.
[16] Ning, J., Zhang, L., Zhang, D., & Wu, C. (2010). Interactive image segmentation by maximal similarity based region merging. Pattern Recognition, 43(2), 445-456.
[17] H. Zhou, J. Zheng, and L. Wei, “Texture aware image segmentation using graph cuts and active contours,” Pattern Recognition, vol. 46, no. 6, pp. 1719-1733, 2013.
[18] N. Houhou, J.-P. Thiran, and X. Bresson, “Fast texture segmentation based on semi-local region descriptor and active contour,” Numerical Mathematics: Theory, Methods and Applications., vol. 2, no. article, pp. 445-468, 2009.
[19] Z. Guo, L. Zhang, and D. Zhang, “A completed modeling of local binary pattern operator for texture classification,” IEEE transactions on image processing, vol. 19, no. 6, pp. 1657-1663, 2010.
[20] L. Liang, C. Liu, Y.-Q. Xu, B. Guo, and H.-Y. Shum, “Real-time texture synthesis by patch-based sampling,” ACM Transactions on Graphics (ToG), vol. 20, no. 3, pp. 127-150, 2001.
[21] N. Sochen, R. Kimmel, and R. Malladi, “A general framework for low level vision,” IEEE Transactions on Image Processing, vol. 7, no. 3, p. 310-318, 1998.
[22] Wang, G., Zuluaga, M. A., Pratt, R., Aertsen, M., Doel, T., Klusmann, M., David, A. L., Deprest, J., Vercauteren, T. and Ourselin, S. [2016b]. Slic-seg: A minimally interactive segmentation of the placenta from sparse and motion-corrupted fetal mri in multiple views, Medical image analysis 34: 137-147.

1. A computer-implemented method for segmentation of multi-energy CT data, the multi-energy CT data including data in three or more energy bands, the method including:
a) receiving in memory the multi-energy CT data;
b) displaying the multi-energy CT data to a user;
c) receiving user input of one or more region indicators for the displayed data;
d) based on the region indicators, generating one or more pixel probability maps; and
e) based on the one or more pixel probability maps, segmenting the multi-energy CT data.

2. The method of claim 1, wherein at least one of the one or more pixel probability maps is generated using a decision tree learning model.

3. (canceled)

4. (canceled)

5. The method of claim 1, further including refining at least one of the one or more probability maps before segmenting the multi-energy CT data.

6. The method of claim 5 wherein refining includes adjusting the probability values for at least some pixels based on one or more distance metrics, wherein the one or more distance metrics are indicative of one or more distances between that particular pixel and one or more of the region indicators.

7. (canceled)

8. The method of claim 6 wherein the one or more distances include one or more of: a Euclidean distance, and a Geodesic distance.

9. (canceled)

10. (canceled)

11. The method of claim 1 wherein each pixel probability map defines, for each pixel, a probability of that pixel being in a data region.

12. The method of claim 1, wherein segmenting the CT data includes labelling the pixels as belonging to different regions.

13. (canceled)

14. The method of claim 1, wherein segmenting the CT data is performed using a conditional random field segmentation method.

15. The method of claim 1 wherein segmenting the CT data includes:
performing an initial segmentation of the CT data, including identifying one or more boundaries between regions; and
refining the initial segmentation of the CT data by further analysis of data around the identified one or more boundaries.

16. (canceled)

17. The method of claim 15 wherein the initial segmentation is performed using a conditional random field segmentation method with a first neighbour connectivity, and the refinement of the initial segmentation is performed using a conditional random field segmentation method with a second neighbour connectivity higher than the first neighbour connectivity.

18. The method of claim 15 wherein the initial segmentation is performed using an alpha-expansion algorithm, and the refinement of the initial segmentation is performed by creating a dense graph using pixels around the identified one or more boundaries and refining the initial segmentation using alpha-expansion optimization.

19. A computer-implemented method for segmentation of medical image data, the method including:
a) receiving in memory the medical image data;
b) displaying the medical image data to a user;
c) receiving user input of one or more region indicators for the displayed data;
d) based on the region indicators, generating one or more pixel probability maps;
e) refining at least one of the one or more probability maps by adjusting probability values for at least some pixels based on two or more distance metrics, wherein, for a particular pixel, the two or more distance metrics are indicative of two or more distances between that particular pixel and one or more of the region indicators, and wherein the two or more distances include a Euclidean distance and a geodesic distance; and
f) based on the refined pixel probability maps, segmenting the medical image data.

20. The method of claim 19, wherein at least one of the one or more pixel probability maps is generated using a decision tree learning model.

21. (canceled)

22. (canceled)

23. (canceled)

24. The method of any one of claim 19 wherein each pixel probability map defines, for each pixel, a probability of that pixel being in a data region.

25. The method of any one of claim 19, wherein segmenting the image data includes labelling the pixels as belonging to different regions.

26. (canceled)

27. The method of claim 19, wherein segmenting the image data is performed using a conditional random field segmentation method.

28. The method of claim 19 wherein segmenting the image data includes:
performing an initial segmentation of the image data, including identifying one or more boundaries between regions; and
refining the initial segmentation of the image data by further analysis of data around the identified one or more boundaries.

29. (canceled)

30. (canceled)

31. (canceled)

32. The method of claim 19 wherein the medical image data includes data captured from a human or animal subject.

33. (canceled)

34. The method of claim 19, including:
a. generating a plurality of 2D slices from the 3D data;
b. selecting one of the 2D slices as a start slice, wherein the received region indicators are in the start slice and the segmentation produces a segmented start slice;
c. propagating data to one or more adjacent further slices;
d. based in part on the propagated data, segmenting the one or more further slices;
e. repeating the propagating of data and segmentation of further slices until the segmented start slice and segmented further slices encompass a desired 3D space; and
f. combining the segmented slices to form segmented 3D data.

35. (canceled)

36. (canceled)

37. (canceled)

38. (canceled)

39. A multi-energy CT system, including:
a multi-energy CT scanner configured to scan a subject to produce multi-energy CT data including data in three or more energy bands;
memory arranged to store the multi-energy CT data;
a display arranged to display the multi-energy CT data to a user;
a user input device arranged for user input of one or more region indicators for the displayed data; and
a processor arranged to:
a) based on the region indicators, generate one or more pixel probability maps;
b) based on the one or more pixel probability maps, segment the multi-energy CT data.

40. (canceled)
